SEQUENCE LISTING (12 sequences)

SEQ ID NO: 1 - protein, 6 aa, synthetic construct
  CARBOHYD 6: note = Glycosylated with D-lactose
  SITE 2: note = D-Lysine
  SITE 2..5: note = Sidechain amino group of D-Lysine is linked via an amide bond to sidechain carboxyl of Glu
  REGION 2..5: note = Peptide is circular
YKWFES

SEQ ID NO: 2 - skipped

SEQ ID NO: 3 - protein, 6 aa, synthetic construct
  SITE 2: note = D-Lysine
  MOD_RES 1: note = (O-tert-butyl ether-N-t-butyloxycarbonyl) L-Tyr
  MOD_RES 5: note = (Allyl ester)Glu
  MOD_RES 2: note = (6N - allyoxycarbonyl)D-Lys
  MOD_RES 6: note = Ser(heptaacetyl-D-lactose)
YKWFES

SEQ ID NO: 4 - protein, 6 aa, synthetic construct
  SITE 2: note = D-Lysine
  MOD_RES 1: note = (O-tert-butyl ether-N-t-butyloxycarbonyl) L-Tyr
  SITE 2..5: note = Sidechain amino group of D-Lysine is linked via an amide bond to sidechain carboxyl of Glu
  REGION 2..5: note = Peptide is circular
  MOD_RES 6: note = Ser(heptaacetyl-D-lactose)
YKWFES

SEQ ID NO: 5 - protein, 6 aa, synthetic construct
  CARBOHYD 6: note = Glycosylated with D-Glucose
  SITE 2: note = D-Lysine
  SITE 2..5: note = Sidechain amino group of D-Lysine is linked via an amide bond to sidechain carboxyl of Glu
  REGION 2..5: note = Peptide is circular
YKWFES

SEQ ID NO: 6 - protein, 7 aa, synthetic construct
  CARBOHYD 7: note = Glycosylated with D-Glucose
  SITE 2: note = D-Lysine
  REGION 2..5: note = Peptide is circular
  SITE 2..5: note = Sidechain amino group of D-Lysine is linked via an amide bond to sidechain carboxyl of Glu
YKWFEGS

SEQ ID NO: 7 - protein, 6 aa, synthetic construct
  CARBOHYD 6: note = Glycosylated with D-Glucose
  SITE 2: note = D-Lysine
  REGION 2..5: note = Peptide is circular
  SITE 2..5: note = Sidechain amino group of D-Lysine is linked via an amide bond to sidechain carboxyl of Glu
  MOD_RES 3: note = D-1-naphthylalanine
YKXFES

SEQ ID NO: 8 - protein, 6 aa, synthetic construct
  CARBOHYD 6: note = Glycosylated with D-Lactose
  SITE 2: note = D-Lysine
  REGION 2..5: note = Peptide is circular
  SITE 2..5: note = Sidechain amino group of D-Lysine is linked via an amide bond to sidechain carboxyl of Glu
  MOD_RES 3: note = D-1-naphthylalanine
YKXFES

SEQ ID NO: 9 - protein, 6 aa, synthetic construct
  CARBOHYD 6: note = Glycosylated with D-Glucose
  SITE 2: note = D-Lysine
  REGION 2..5: note = Peptide is circular
  SITE 2..5: note = Sidechain amino group of D-Lysine is linked via an amide bond to sidechain carboxyl of Glu
  MOD_RES 3: note = 1-naphthylalanine
YKXFES

SEQ ID NO: 10 - protein, 7 aa, synthetic construct
  CARBOHYD 6..7: note = Glycosylated with D-Glucose
  SITE 2: note = D-Lysine
  REGION 2..5: note = Peptide is circular
  SITE 2..5: note = Sidechain amino group of D-Lysine is linked via an amide bond to sidechain carboxyl of Glu
YKWFESS

SEQ ID NO: 11 - protein, 6 aa, synthetic construct
  SITE 2: note = D-Lysine
  SITE 2..5: note = Sidechain amino group of D-Lysine is linked via an amide bond to sidechain carboxyl of Glu
  REGION 2..5: note = Peptide is circular
YKWFEG

SEQ ID NO: 12 - protein, 6 aa, synthetic construct
  SITE 2: note = D-Lysine
  REGION 2..5: note = Peptide is circular
  SITE 2..5: note = Sidechain amino group of D-Lysine is linked via an amide bond to sidechain carboxyl of Glu
YKWFES

(19) United States
(12) Patent Application Publication
(10) Pub. No.: US 2025/0296958 A1
(43) Pub. Date: Sep. 25, 2025

(54) GLYCOSYLATED CYCLIC ENDOMORPHIN ANALOGS

(71) Applicants: THE ADMINISTRATORS OF THE TULANE EDUCATIONAL FUND, New Orleans, LA (US); DEPARTMENT OF VETERANS AFFAIRS (US), Washington, DC (US); ARIZONA BOARD OF REGENTS on behalf of the UNIVERSITY OF ARIZONA, Tucson, AZ (US)

(72) Inventors: James E. ZADINA, Metaire, LA (US); Robin POLT, Tucson, AZ (US); Lajos SZABO, Tucson, AZ (US)

(21) Appl. No.: 18/869,591

(22) PCT Filed: May 30, 2023
(86) PCT No.: PCT/US23/23879

§ 371 Date: Nov. 26, 2024

Related U.S. Application Data
(60) Provisional application No. 63/347,182, filed on May 31, 2022.

(51) Int. Cl. C07K 7/64; A61K 38/00; A61P 25/04

(52) U.S. Cl. CPC C07K 7/64; A61P 25/04; A61K 38/00

(57) ABSTRACT

Glycosylated, cyclic peptides of Formula (I): A¹-cyclo[A²-A³-A⁴-A⁵]-A⁶-O-Carb, Formula (II): A¹-cyclo[A⁵-A³-A⁴-A²]-A⁶-O-Carb, and pharmaceutically acceptable salts thereof, are described herein, which are useful, e.g., in treating pain. In some embodiments A¹ is L-Tyr; A² is a D-Lys, D-Orn, D-Dab, or D-Dpr; A³ is L-Trp; A⁴ is L-Phe, A⁵ is an amino acid residue selected from the group consisting of Asp, Glu, iso-Asp, and iso-Glu; A⁶ is (a) a hydroxy-substituted amino acid residue (HO-AA), or (b) an oligopeptide comprising 2 to 5 amino acid residues comprising the HO-AA; Carb is a carbohydrate group bonded to the sidechain oxygen of the HO-AA by a β3-D-glycosidic bond, and the C-terminus of A⁶ optionally is amidated, e.g., as a primary amide.

### CROSS-REFERENCE TO RELATED APPLICATIONS

This application claims the benefit of U.S. Provisional Application Serial No. 63/347,182, filed on May 31, 2022, which is incorporated herein by reference in its entirety.

### STATEMENT REGARDING FEDERALLY SPONSORED RESEARCH OR DEVELOPMENT

This invention was made with government support under I01BX003776 awarded by the United States Department of Veterans Affairs, and DA052539 awarded by the National Institutes of Health. The government has certain rights in this invention.

### INCORPORATION OF SEQUENCE LISTING

Biological sequence information for this application is included in a XML file having the file name “TU-685 PCT.xml”, created on May 24, 2023, and having a file size of 29,122 bytes, which is incorporated herein by reference.

### FIELD OF THE INVENTION

This invention relates to new opioid receptor agonists for use in methods for treating pain and opioid use disorder, among others. More particularly, this invention relates to glycosylated cyclic endomorphin-1 (EM1) and endomorphin-2 (EM2) analogs and methods of treatment using the analogs.

### BACKGROUND

Opioids acting at the mu opioid receptor (MOR) remain the gold standard for moderate to severe pain relief, but serious side effects limit their use, particularly abuse liability. Concern with inadequately treated pain, balanced with fear of addiction, has led to pendulums of increased opioid prescribing followed by increased restrictions. The opioid overdose epidemic, however, has grown steadily in roughly three waves: a steady growth since the 1990's with increased opioid prescriptions, sharp increases beginning in 2010 with heroin overdoses and, recently with increases in fentanyl overdoses (CDC1). According to the Centers for Disease Control (CDC), opioids were involved in 49,860 overdose deaths or 137 deaths/day in 2019, which represents 70.6% of all drug overdose deaths (CDC2), which is more than the National Highway Traffic Safety Administration (NHTSA) reports occurs in auto accidents (93/day; NHTSA). While 38 of these deaths per day (28%) are directly tied to prescription drugs, a recent study showed that, of those who began abusing opioids in the 2000s, 75 percent reported that their first opioid was a prescription drug (Cicero et al. 2014). Limitations of drugs available for treatment of pain play a crucial role in the epidemic as indicated by a CDC suggestion (CDC1): “To reverse this epidemic, we need to improve the way we treat pain. We must prevent abuse, addiction, and overdose before they start”. Thus, a compound that provides effective pain relief without addictive properties may help prevent the initiation of addiction and serve as a treatment for it.

Currently available treatments for OUD include methadone, buprenorphine, naltrexone, and buprenorphine+naloxone in various forms. Methadone and buprenorphine have played a valuable role in the treatment of OUD. They produce effective opioid substitution effects with relatively long durations of action that can reduce the need for subsequent doses. They do, however, retain reward properties. They are tightly regulated because they have their own propensity for abuse indicated by robust intravenous self-administration (SA) rates, locomotor sensitization, and conditioned place preference (CPP) behaviors in rats (Martin et al. 2007; Steinpreis, Rutell, and Parrett 1996; Tzschentke 2004; Wade et al. 2015). In humans, buprenorphine and methadone have clinical utility for reducing the positive subjective effects of opioids, but both compounds are self-administered and produce positive reinforcing effects (Comer, Sullivan, and Walker 2005; Jones, Madera, and Comer 2014). Buprenorphine is combined with the antagonist naloxone in several formulations such as Suboxone, Bunavail, and Zubsolv, all of which are susceptible to inducing withdrawal effects. Naltrexone, a full opioid antagonist, can block opioid cravings, but precipitates withdrawal symptoms and cannot be used prior to a medically supervised opioid withdrawal. Thus, the currently available treatments have had successes, but also serious limitations, and novel treatments are urgently needed. Novel therapies with reduced reward properties could therefore increase the armamentarium of options for treatment and management of OUD.

A drug that effectively treats pain without rewarding effects could play a critical role in reducing OUD. Such a compound could also serve as a treatment for OUD. The mu opioid receptor is the target for the most successful current treatments for OUD, including the mu agonist methadone and the partial agonist buprenorphine. These materials have had success but are themselves subject to abuse, produce positive reinforcing effects and are subject to withdrawal symptoms. A substitution therapy with low or absent abuse liability could transform treatment for OUD.

MOR was recently designated as a Tain (clinic) protein in the target development level (TDL) classification by the Illuminating the Druggable Genome (IDG) Knowledge Management Center (Oprea et al. 2018). This designation reflects targets linked to at least one approved drug/active pharmaceutical ingredient by mechanism of action (MoA). MOR is one of the most extensively studied drug targets, and this has led to a large knowledge base enabling numerous avenues of approach for separating the desired (analgesic) from undesired effects mediated by the receptor. Multiple mechanisms of action are now known to be mediated through MOR, including G-protein and β-arrestin activation, ion channel regulation and numerous intracellular signaling functions (Al-Hasani and Bruchas 2011). Bohn and co-workers showed that β-arrestin knockout mice displayed enhanced antinociception and reduced respiratory depression and GI dysfunction by morphine (Bohn et al. 1999; Raehal, Walker, and Bohn 2005). These findings raised the possibility that the gold standard analgesia produced by MOR activation could be achieved with fewer side effects based on differential activation of the various mechanisms mediated by MOR.

Numerous programs have sought to develop biased agonists—compounds that selectively activate one signaling pathway over another—in this case, G-protein over β-arrestin signaling. Indeed, this approach has successfully produced compounds that increase antinociception while reducing respiratory depression and GI dysfunction (e.g., Schmid et al. 2017). However, a finding particularly important for OUD is that β-arrestin knockout mice and G-protein biased agonists also displayed an increased sensitivity to the rewarding effects of morphine that included increased CPP and striatal dopamine release compared to wild-type mice (Bohn et al. 2003) and changes in intracranial self-stimulation (Altarifi et al. 2017). In addition, G-protein biased agonists show increased hyperalgesia (Araldi, Ferrari, and Levine 2018) and β-arrestin knockout mice show increased allodynia (Chen et al. 2016). These studies indicate that G-protein/β-arrestin biased agonists may not improve, and could exacerbate, abuse liability and duration of pain. Thus, while biased agonists reflect the promise of eventually separating desired from undesired effects, much work remains to meet this promise regarding OUD.

Endomorphins (EMs) are potent and selective natural short peptide agonists for the mu opioid receptor (MOR), the main analgesic target for currently used opioids such as morphine. Shortly after their discovery (Zadina et al. 1997), the EMs showed a promising profile of potent analgesia with some reduced side effects, including reduced reward (Wilson et al. 2000) and respiratory depression (Czapla et al. 2000) for EM1. Because the natural peptides are unstable in plasma, medications based on the EMs require chemical modification of the structure (EM analogs). Cyclized, D-amino acid-containing EM analogs have been described by Zadina et al. which were evaluated for (1) metabolic stability for favorable drug properties, (2) highly effective antinociception, and (3) significant reduction of adverse side effects. After extensive screening of numerous analogs for these properties, four that showed considerable promise were characterized in depth (Zadina et al. 2016; and U.S. Pat. Nos. 8,716,436 and 10,919,939). While these materials have shown some useful properties, particularly the EM1 analog referred to as ZH853, there is still room for improvement, e.g., to improve blood brain barrier and blood gut barrier penetration.

Antinociceptive studies indicated that ZH853 can penetrate key barriers including the gut-blood barrier, leading to oral effectiveness, and the blood brain barrier (BBB), indicating central activation. However, there are important limitations that clearly indicate that these analogs will require formulation or alteration to improve central penetration and be reliably effective as an oral formulation. This is particularly important for potential treatment of substance abuse disorder, since the injection route could provide a relapse cue.

For example, in pain tests comparing ZH853 to morphine (Feehan et al. 2017), the ED₅₀ for pain relief was similar for morphine and ZH853 after peripheral (i.v.) administration. The average ED₅₀ in neuropathic, inflammatory and postoperative pain tests was 1.4-fold higher for ZH853 relative to morphine on a mass basis (mg/kg) and 1.7-fold lower on a molar basis, indicating a morphine equivalence of about 1. By contrast, intrathecal doses of ZH853 for the three models were on average 62-fold more potent than morphine. These data indicate that less ZH853 penetrates to central tissue than morphine, but that the analog is considerably more potent in activating these targets, resulting in the approximately equal potency intravenous (i.v.) effects. Thus, materials with improved barrier penetration could substantially lower the dose requirements and limit peripherally-mediated side effects. In addition, greater oral effectiveness would provide a preferable route of administration for pain and OUD treatments.

In addition, compounds that selectively bind to the delta and kappa opioid receptors (DOR and KOR, respectively) also have been reported as useful in treating pain and other conditions, such as drug dependence, although with limited success.

In view of the issues with current opioid compounds and methods of treating pain (e.g., chronic pain, neuropathic pain, inflammatory pain, and the like), drug dependence, and OUD, there is an ongoing need for new opioid compounds. The compounds and methods described herein address this need.

### SUMMARY

The compounds described herein are cyclic peptide analogs of EM1 and EM2, which have a cyclic peptide pharmacophore flanked by a tyrosine or tyrosine derivative at the N-terminus and a glycosylated amino acid or a glycosylated short peptide chain at the C-terminus. In some embodiments, the EM analogs are peptides of Formula (I): A¹-cyclo[A²-A³-A⁴-A⁵]-A⁶-O-Carb, or Formula (II): A¹-cyclo[A⁵-A³-A⁴-A²]-A⁶-O-Carb; and pharmaceutically acceptable salts thereof. These compounds can be used in a method for treating pain (e.g., chronic pain, neuropathic pain, inflammatory pain, and the like), as well as in methods for treating drug dependence and OUD. The innovative approach to the development of pain medications described herein, departs from the strategies of modifying compounds mostly derived from opium, including one hundred-year old compounds like oxycodone and hydrocodone. The latter, in combination with acetaminophen (e.g., Vicodin), was the most widely prescribed of all drugs in the US in 2013 (Informatics 2014). In contrast, the pharmacophores utilized herein are glycosylated cyclic analogs of EM1 that are similar to some of the materials described by Zadina et al. (Zadina et al. 2016; and U.S. Pat. Nos. 8,716,436 and 10,919,939, which are incorporated herein by reference in their entireties).

As used herein, the portions peptide formulas illustrated by “cyclo[ . . . ]” and “c[ . . . ]” refer to cyclic peptides in which the peptide ring is formed by a non-peptidyl cross-linking amide bond between the first and last amino acid residues within the square brackets.

For reference, the abbreviations for amino acids described herein include alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), valine (Val), ornithine (Orn), 1-naphthylalanine (Nal), 2,3-diaminopropionic acid (Dpr), 2,4-diaminobutyric acid (Dab), allo-threonine (allo-Thr), cis-4-hydroxy proline (cis-4-hydroxy-Pro), trans-4-hydroxy proline (trans-4-hydroxy-Pro), cis-3-hydroxy proline (cis-3-hydroxy-Pro), trans-3-hydroxy proline (trans-3-hydroxy-Pro), homo glutamic acid (hGlu; also referred to as 2-amino-1,6-hexanedioic acid), homo lysine (hLys; also referred to as 2,7-diaminoheptanoic acid), bis-homo glutamic acid (bhGlu; also referred to as 2-amino-1,7 heptanedioic acid), and bis-homo lysine (bhLys; also referred to as 2,8-diaminooctanoic acid).

Many examples of natural and unnatural amino acids are described in Chapter 14 “Amino Acid Properties and Consequences of Substitutions” by Matthew J. Betts and Robert B. Russell in Bioinformatics for Geneticists, Matthew R. Barns and Ian C. Cray, Eds. John Wiley & Sons, Ltd, pp. 298-316 (2003), hereinafter referred to as Betts and Russell, which is incorporated herein by reference in its entirety.

As used herein, “isoAsp”, “isoGlu”, “isohGlu”, and “isobhGlu”, respectively refer to aspartic acid, glutamic acid, homoglutamic acid, and bis-homoglutamic acid residues in a peptide chain that are bonded to the adjacent following amino acid residue in the chain (going in the direction from N-terminus to C-terminus) by an amide bond between the sidechain carboxyl of the aspartic acid, glutamic acid, homglutamic acid or bis-homoglutamic acid, and the α-amino group of the following amino acid residue. In other words, the alpha carboxyl group of an isoAsp, isoGlu, or isohGlu residue does not form part of the peptide backbone.

As used herein, the term “hydroxy-substituted amino acid” (“HO-AA”) refers to an amino acid having a hydroxyl substituent on the sidechain thereof. Non-limiting examples of hydroxy-substituted amino acids include Ser, Thr, allo-Thr, homoSer, cis-4-hydroxy-Pro, trans-4-hydroxy-Pro, cis-3-hydroxy-Pro, and trans-3-hydroxy-Pro.

In Formula (I) and Formula (II):
A¹ is an amino acid residue selected from the group consisting of Tyr, N-methyl-Tyr, 2,6-dimethyl-Tyr, and N-methyl-2,6-dimethyl-Tyr;
A² is an amino acid residue selected from the group consisting of Lys, hLys, bhLys, Orn, Dab, and Dpr;
A³ is an amino acid residue selected from the group consisting of Trp, N-methyl-Trp, Phe, N-methyl-Phe, p-X-Phe, and N-methyl-p-X-Phe;
A⁴ is an amino acid residue selected from the group consisting of Phe, N-methyl-Phe, p-X-Phe, and N-methyl-p-X-Phe; wherein X is F, Cl, Br, or NO₂ at the 4-position of the phenyl group of the Phe sidechain;
A⁵ is an amino acid residue selected from the group consisting of Asp, Glu, hGlu, bhGlu, isoAsp, isoGlu, isohGlu, and isobhGlu;
A⁶ is (a) a hydroxy-substituted amino acid residue (HO-AA) (e.g., a HO-AA selected from the group consisting of Ser, Thr, homoSer, cis-4-hydroxy-Pro, trans-4-hydroxy-Pro, cis-3-hydroxy-Pro, and trans-3-hydroxy-Pro); or (b) an oligopeptide comprising 2 to 5 amino acid residues (e.g., a dipeptide, a tripeptide, a tetrapeptide or a pentapeptide) comprising at least one HO-AA;
Carb is a carbohydrate group such as, e.g., glucose (D-Glc), lactose (D-Lact, or D-Glc-β-D-Gal), and the like, which is bonded to sidechain oxygen of the HO-AA of A⁶ by a β-D-glycosidic bond;
the notations cyclo[A²-A³-A⁴-A⁵] and cyclo[A⁵-A³-A⁴-A²] in Formulas (I) and (II) represent a cyclic peptide ring in which the sidechain amino group of A² is bonded to A⁵ by an amide bond, thereby forming a ring in which, when A⁵ is isoAsp, isoGlu, isohGlu, or isobhGlu, the sidechain amino group of A² is bound to the α-carboxyl group of A⁵ by an amide bond, whereas when A⁵ is Asp, Glu, hGlu or bhGlu, the sidechain amino group of A² is bound to the sidechain carboxyl group of A⁵ by an amide bond; and
optionally, the C-terminus of A⁶ is amidated (e.g., as a primary amide).

In some embodiments, the N-terminal residue of the oligopeptide is the HO-AA.

In some other embodiments, the C-terminal residue of the oligopeptide is an HO-AA. In yet other embodiments, the oligopeptide comprises two or more HO-AA residues. In some preferred embodiments, the C-terminus of A⁶, is amidated as a primary amide.

In addition to the HO-AA, the oligopeptide can comprise any combination of amino acid residues, including e.g., D-amino acid residues, L-amino acid residues and Gly.

As used herein, the term EM1 analog refers to peptides in which A³=Trp, or N-methyl-Trp, while the term EM2 analog refers to peptides in which A³=Phe, N-methyl-Phe, p-X-Phe, or N-methyl-p-X-Phe.

In some preferred embodiments of Formula (I), A² is a D-amino acid residue.

In some preferred embodiments of Formula (II), A⁵ is a D-amino acid residue, particularly when A⁵ is Asp, Glu, hGlu, or bhGlu.

The compounds of Formula (I) and Formula (II) are useful for treating pain, drug dependence and opioid use disorder.

In some preferred embodiments, the peptides are cyclic peptides of Formula (I).

In some embodiments, a pharmaceutical composition comprises a glycosylated peptide of Formula (I) and/or Formula (II), or a pharmaceutically acceptable salt thereof, in a pharmaceutically acceptable carrier.

In some embodiments, a method of treating pain comprises administering a glycosylated peptide of Formula (I) and/or Formula (II), or a pharmaceutically acceptable salt thereof, in a pharmaceutically acceptable carrier to a patient in need thereof (i.e., a patient suffering from pain, including but not limited to chronic pain, neuropathic pain, and inflammatory pain).

In some embodiments, a method of treating drug dependence comprises administering a glycosylated peptide of Formula (I) and/or Formula (II), or a pharmaceutically acceptable salt thereof in a pharmaceutically acceptable carrier to a patient in need thereof (i.e., a patient who is dependent upon or addicted to a drug, such as an opioid drug.

In another embodiment, a method for treating opioid use disorder comprises administering to a subject in need thereof a pharmaceutical composition comprising a glycosylated peptide of Formula (I) and/or Formula (II), or a pharmaceutically acceptable salt thereof in a pharmaceutically acceptable carrier. For example, the peptide can be administered in place of, and as a substituted for a mu opioid receptor agonist to which the subject is addicted. In some embodiments, the subject will be addicted to one or more opioid such as, e.g., morphine, oxycodone, hydrocodone, codeine, heroin, and the like, e.g., to block withdrawal symptoms induced by discontinuing chronic exposure to the opioid to which the subject is addicted. Often, the subject will have been previously treated for OUD using a drug such as methadone, buprenorphine, naltrexone, and the like.

In some embodiments for treating pain, drug dependence, or OUD, the subject will be treated intravenously with a glycosylated peptide of Formula (I) and/or Formula (II), or a pharmaceutically acceptable salt thereof. In other embodiments, the subject will be treated orally with the glycosylated peptide. In treating OUD, initial doses of the cyclic peptide may be at a low dose such as a dose that is less than the ED50 for the peptide for analgesia. In some embodiments, the treatment will begin at the low dose and will be increased over time to a higher maintenance level during the course of the treatment.

Without wishing to be bound by theory, it is believed that glycosylation at or near the C-terminus of the cyclic endomorphin analogs modulates the membrane affinity of the pharmacophore, allowing it to “hop” from membrane to membrane, and to traverse cellular barriers (i.e. the blood-brain barrier) so that the drug can reach mu receptors in the brain and elsewhere. Together with cyclization, glycosylation leads to more effective analgesia mediated by mu receptors in the brain.

The following non-limiting embodiments are described below to illustrate certain features and aspects of the compositions and methods described herein.

Embodiment 1 is a glycosylated, cyclic peptide of Formula (I): A¹-cyclo[A²-A³-A⁴-A⁵]-A⁶-O-Carb, Formula (II): A¹-cyclo[A⁵-A³-A⁴-A²]-A⁶-O-Carb, and pharmaceutically acceptable salts thereof,

wherein:
A¹ is an amino acid residue selected from the group consisting of Tyr, N-methyl-Tyr, 2,6-dimethyl-Tyr, and N-methyl-2,6-dimethyl-Tyr;
A² is an amino acid residue selected from the group consisting of Lys, hLys, bhLys, Orn, Dab, and Dpr;
A³ is an amino acid residue selected from the group consisting of Trp, N-methyl-Trp, Phe, N-methyl-Phe, p-X-Phe, and N-methyl-p-X-Phe;
A⁴ is an amino acid residue selected from the group consisting of Phe, N-methyl-Phe, p-X-Phe, and N-methyl-p-X-Phe;
X is F, Cl, Br, or NO₂ at the 4-position of the phenyl group of the Phe sidechain;
A⁵ is an amino acid residue selected from the group consisting of Asp, Glu, homoGlu, bishomoGlu, isoAsp, isoGlu, isohomoGlu, and isobishomoGlu;
A⁶ is an amino acid residue or an oligopeptide comprising 2 to 5 amino acid residues, and A⁶ comprises at least one hydroxy-substituted amino acid residue;
Carb is a carbohydrate group bonded to a sidechain oxygen of the HO-AA by a β-D-glycosidic bond;
cyclo[A²-A³-A⁴-A⁵] and cyclo[A²-A³-A⁴-A⁵] each represent a cyclic peptide ring in which A² is bonded to A⁵; wherein when A⁵ is isoAsp, isoGlu, isohomoGlu, or isobishomoGlu, the sidechain amino group of A² is bonded the α-carboxyl group of A⁵ by an amide bond; whereas when A⁵ is Asp, Glu, homoGlu or bishomoGlu, the sidechain amino group of A² is bonded to the sidechain carboxyl group of A⁵ by an amide bond; and
wherein the C-terminal carboxyl group of A⁶ optionally is amidated.

Embodiment 2 is the peptide of embodiment 1, wherein A¹ is Tyr.

Embodiment 3 is the peptide of embodiment 1, wherein A¹ is N-methyl-Tyr.

Embodiment 4 is the peptide of any one of embodiments 1 to 3, wherein A³ is Trp.

Embodiment 5 is the peptide of any one of embodiments 1 to 3, wherein A³ is N-methyl-Trp.

Embodiment 6 is the peptide of any one of embodiments 1 to 3, wherein A³ is Phe.

Embodiment 7 is the peptide of any one of embodiments 1 to 6, wherein A⁴ is Phe.

Embodiment 8 is the peptide of any one of embodiments 1 to 6, wherein A⁴ is N-methyl-Phe.

Embodiment 9 is the peptide of any one of embodiments 1 to 6, wherein A⁴ is p-X-Phe, wherein X is F, Cl, Br, or NO₂ at the 4-position of the phenyl group of the Phe sidechain.

Embodiment 10 is the peptide of any one of embodiments 1 to 6, wherein A⁴ is N-methyl-p-X-Phe, wherein X is F, Cl, Br, or NO₂ at the 4-position of the phenyl group of the Phe sidechain.

Embodiment 11 is the peptide of embodiment 1, wherein A¹ is Tyr, A³ is Trp, and A⁴ is Phe.

Embodiment 12 is the peptide of any one of embodiments 1 to 11, wherein A⁵ is L-Asp.

Embodiment 13 is the peptide of any one of embodiments 1 to 11, wherein A⁵ is L-Glu.

Embodiment 14 is the peptide of any one of embodiments 1 to 11, wherein A⁵ is D-Asp.

Embodiment 15 is the peptide of any one of embodiments 1 to 11, wherein A⁵ is D-Glu.

Embodiment 16 is the peptide of any one of embodiments 1 to 11, wherein A⁵ is L-isoAsp.

Embodiment 17 is the peptide of any one of embodiments 1 to 11, wherein A⁵ is L-isoGlu.

Embodiment 18 is the peptide of any one of embodiments 1 to 11, wherein A⁵ is D-isoAsp.

Embodiment 19 is the peptide of any one of embodiments 1 to 11, wherein A⁵ is D-isoGlu.

Embodiment 20 is the peptide of any one of embodiments 1 to 19, wherein A⁶ is L-Ser.

Embodiment 21 is the peptide of any one of embodiments 1 to 19, wherein A⁶ is L-Ser-NH₂.

Embodiment 22 is the peptide of any one of embodiments 1 to 19, wherein A⁶ is L-Thr.

Embodiment 23 is the peptide of any one of embodiments 1 to 19, wherein A⁶ is L-Thr-NH₂.

Embodiment 24 is the peptide of any one of embodiments 1 to 23, wherein Carb is β-D-glucose.

Embodiment 25 is the peptide of any one of embodiments 1 to 23, wherein Carb is β-D-lactose.

Embodiment 26 is the peptide of any one of embodiments 1 to 25, where in the peptide is a cyclic peptide of Formula (I).

Embodiment 27 is the peptide of any one of embodiments 1 to 25, where in the peptide is a cyclic peptide of Formula (II).

Embodiment 28 is the peptide of embodiment 1 or embodiment 26 which is:

(SEQ ID NO: 1)
Tyr-c[D-Lys-Trp-Phe-Glu]-Ser(β-Lact)-NH₂.

Embodiment 29 is the peptide of embodiment 1 or embodiment 26 which is:

(SEQ ID NO: 5)
Tyr-c[D-Lys-Trp-Phe-Glu]-Ser(β-Glc)-NH₂.

Embodiment 30 is the peptide of embodiment 1 or embodiment 26 which is:

(SEQ ID NO: 10)
Tyr-c[D-Lys-Trp-Phe-Glu]-Ser(β-Glc)-Ser(β-Glc)-NH₂.

Embodiment 31 is a pharmaceutical composition comprising the peptide of any one of embodiments 1 to 30 or a pharmaceutically acceptable salt thereof in a pharmaceutically acceptable carrier.

Embodiment 32 is a method of treating pain comprising administering to a subject in need thereof the pharmaceutical composition of embodiment 31.

Embodiment 33 is the method of embodiment 32, wherein the pain is chronic pain.

Embodiment 34 is the method of embodiment 32 or 33, wherein the pain is neuropathic pain.

Embodiment 35 is the method of any one of embodiments 32 to 34, wherein the pain is inflammatory pain.

Embodiment 36 is a method for treating a drug dependence comprising administering to a subject in need thereof a therapeutically effective amount of the pharmaceutical composition of embodiment 31.

Embodiment 37 is a method for treating opioid use disorder comprising administering to a subject in need thereof a therapeutically effective amount of the pharmaceutical composition of embodiment 31.

Embodiment 38 is the method of embodiment 37, wherein the composition is administered in place of, and as a substitute for, a mu opioid receptor agonist to which the subject is addicted.

Embodiment 39 is the peptide of any one of embodiments 1 to 30 for treating pain.

Embodiment 40 is the peptide of embodiment 39, wherein the pain is chronic pain.

Embodiment 41 is the peptide of embodiment 39 or 40, wherein the pain is neuropathic pain.

Embodiment 42 is the peptide of any one of embodiments 39 to 41, wherein the pain is inflammatory pain.

Embodiment 43 is the peptide of any one of embodiments 1 to 30 for treating drug dependence.

Embodiment 44 is the peptide of any one of embodiments 1 to 30 for treating opioid use disorder.

### BRIEF DESCRIPTION OF THE DRAWINGS

FIG. 1 structurally illustrates the difference in ring size between two glycosylated peptides of Formula (I). Panel A illustrates Analog A2, Tyr-c[D-Lys-Trp-Phe-Glu]-Ser(β-Lact)-NH₂ (SEQ ID NO: 1), wherein A² is D-Lys and A⁵ is L-Glu; and Panel B illustrates a peptide of Formula (I) in which A² is D-Dpr and A⁵ is isoGlu, i.e., Tyr-c[D-Dpr-Trp-Phe-isoGlu]-4-OH-Pro(β-D-Glc)-NH₂ (SEQ ID NO: 2).

FIG. 2 schematically illustrates aspects of the synthesis of glycosylated EM1 Analog A2. Panel A illustrates the functional portion of a Rink resin and the order in which acids are added to the peptide. Panel B illustrates the ring closure of the linear peptide of SEQ ID NO: 3 to protected cyclic peptide of SEQ ID NO: 4, as well as deprotection and cleavage of the cyclic peptide from the resin to form Analog A2 (SEQ ID NO: 1).

FIG. 3 illustrates seven glycosylated analogs of EM1 (Analogs A1-A7) and reference compounds ZH853 and A8, which were evaluated herein: A1: SEQ ID NO: 5; A2: SEQ ID NO: 1; A3: SEQ ID NO: 6; A4: SEQ ID NO: 7; A5: SEQ ID NO: 8; A6: SEQ ID NO: 9; and A7: SEQ ID NO: 10; A8 SEQ ID NO: 12; and ZH853: SEQ ID NO: 11.

FIG. 4 provides a graph of receptor binding results for seven glycosylated endomorphin analogs for binding to mu, delta and kappa (μ, δ, κ) opioid receptors (MOR, DOR and KOR, respectively), compared to two non-glycosylated control compounds: ZH853 and A8 having the C-terminal Gly replaced with a Ser (i.e., Tyr-c[D-Lys-Trp-Phe-Glu]-Ser-NH₂; SEQ ID NO: 12), to which the glycosides were coupled in the test compounds. Ki's and selectivity are shown in visual format on a log₂ scale with selectivity values for δ/μ, and κ/μ in columns.

FIG. 5 provides time course results from tail-flick (TF) antinociception tests. Three glycosylated EM analogs were selected for TF testing in male and female CD-1 mice and compared to two reference compounds, ZH853 and morphine. All compounds produced dose-dependent antinociception, expressed as % maximum possible effect (% MPE). The legend shows the doses used (mg/kg s.c.), followed by the total number of animals for the time course and dose-response curves. While values for all doses of the reference compounds returned to baseline within 3 hours, responses to the higher doses of glycopeptides, particularly Analog A2, remained high at the 5-hour limit of the test.

FIG. 6 provides area under the curve (AUC) (panels A and B) and duration (panels C and D) of TF antinociception results. Significant dose-dependent increases in the antinociceptive AUC were observed for the glycopeptides (panel A) and reference compounds (panel B, X-axis scaled as in panel A for reference). Significant main effects of drug showed that Analog A2 produced greater AUC values than all other compounds, that all three glycopeptides showed greater AUC than the reference compounds (p<0.0001), and that significant differences were observed among all compounds with an overall rank order of effectiveness as Analogs A2>A7>A1>morphine>ZH853. In general, female scores were lower than males. For morphine, this resulted in a main effect of sex. However, a dose×sex interaction was observed for the double glycoside Analog A⁷, where female scores were greater than males at the high dose. AUC values for the mean male and female responses to the top dose (5.6 mg/kg) of controls ZH853 and morphine are shown as dashed lines in panels A and C for reference and indicate a minimum of 2.5-fold (>25000 versus (vs) 10000 AUC) to 3-fold (1.8 mg/kg Analog A2>5.6 mg/kg) greater effectiveness of Analog A2 over reference compounds. These differences are conservative as indicated by “>” above bars where responses >50% MPE were maintained at the 5 hour (hr) maximum test time for the top 2 doses of Analog A2. Duration of antinociception >50% MPE (panels C and D) showed similar statistical results but provides a direct index of duration and reflects a minimum 3 to 5-fold greater duration of antinociception relative to controls at the same dose. Panels E and F show that a separate group of male mice (n=6) tested at 10 mg/kg morphine (MS) produced peak effect, time course, duration, and AUC responses (total antinociception) well matched to 1.8 mg/kg A2, indicating that A2 provides antinociception equal to morphine at a dose 5.55-fold lower. Panel F: AUC for matched doses of morphine and A2 expressed as mg/kg and mol/kg. The latter indicates equiantinociceptive doses for A2 are 21.8-fold lower than morphine on a molar basis. “>”=responses >50% MPE maintained at the 5-hr maximum test time. (****, ** p<0.0001, p<0.01, “≥” responses >50% MPE maintained at the 5-hr maximum test time; n values as in FIG. 5 except for 3.2 and 5.6 mg Analog A2 and 5.6 mg of Analog A1 (n=4-5).

FIG. 7 provides TF Dose-Response curves. Panel A provides Percent MPE values at peak response times as described in methods were used to determine the ED₅₀'s. Male and female (M, F) dose-response curves for each compound are shown. Panel B provides tables of ED₅₀ values calculated from doses expressed both as mg/kg (top) and μmol/kg (bottom). The tables provide ED₅₀'s, and relative potencies in male vs female, for each glycopeptide versus both control compounds. Compounds are listed by rank order of potency and show that Analog A2 is 2 to 3-fold more potent than controls for values in units of mg/kg and 4 to 8-fold more potent for values in units of μmol/kg.

FIG. 8 provides time course results from hot plate (HP) antinociception tests. The three analogs and two reference compounds tested in the tail flick test were also tested for HP responses in male and female DBA mice for 5 hr. All compounds produced dose-dependent antinociception, expressed as % maximum possible effect (% MPE). Doses (mg/kg) and n values are shown in the legends.

FIG. 9 provides area under the curve (AUC) and duration of HP antinociception results. As with the TF test, all compounds produced dose-dependent increases in duration of antinociception, as shown for AUC (panels A and B) and duration >50% MPE (panels C and D). Main effects of drug were not significant between A2 and A7, or among A1 and the reference compounds ZH853 and morphine, indicating two tiers of effectiveness, with A2 and A7 producing higher overall antinociceptive effects than the other compounds. Sex differences (and interactions) were not significant, indicating similar effects for both sexes for all compounds in this test. Values for the mean male and female responses to the top dose (5.6 mg/kg) of ZH853 and morphine are shown as dashed lines in panels A and C and indicate about 3-fold greater effectiveness of A2 versus the control compounds (1.8 mg/kg for A2 vs 5.6 mg/kg for controls). Panel E directly illustrates that 1.8 mg/kg of A2 provides a similar peak effect, duration, and AUC compared to 5.6 mg/kg morphine, confirming a 3.1-fold greater effectiveness. (**** p<0.0001, n's as in FIG. 8).

FIG. 10 provides Hot Plate (HP) results as dose-response curves (A) and in tabular form (B). Percent MPE values at peak response times as described in methods were used to determine the ED₅₀'s. Male and female (M, F) dose-response curves for each compound are shown. The tables provide ED₅₀'s and relative potencies of male vs female, for each glycopeptide vs both control compounds. ED₅₀ values in panel B were calculated from doses expressed both as mg/kg (top) and μmol/kg (bottom) are shown. Compounds are listed by rank order of potency and show that A2 and A7 are approximately 2-fold more potent than controls for values in units of mg/kg and 3 to 9-fold for values in units of μmol/kg.

FIG. 11 shows conditioned place preference (CPP) results with 3 equiantinociceptive doses of morphine and A2, providing graphs of both time change on the drug side of the test chamber (left) and area under curve (AUC; right inset) results.

### DETAILED DESCRIPTION

Glycosylated, cyclic EM analogs of Formula (I): A¹-cyclo[A²-A³-A⁴-A⁵]-A⁶-O-Carb, Formula (II): A¹-cyclo[A⁵-A³-A⁴-A²]-A⁶-O-Carb, and pharmaceutically acceptable salts thereof, are described herein. In Formulas (I) and (II), A¹ is an L-amino acid residue selected from the group consisting of Tyr, N-methyl-Tyr, 2,6-dimethyl-Tyr, and N-methyl-2,6-dimethyl-Tyr; A² is a D- or L-amino acid residue selected from the group consisting of Lys, hLys, bhLys, Orn, Dab, and Dpr; A³ is an amino acid residue selected from the group consisting of Trp and N-methyl-Trp; A⁴ is an residue selected from the group consisting of Phe, N-methyl-Phe, p-X-Phe, and N-methyl-p-X-Phe, wherein X is F, Cl, Br, or NO₂ at the 4-position of the phenyl group of the Phe sidechain; and A⁵ is an amino acid residue selected from the group consisting of Asp, Glu, hGlu, bhGlu, iso-Asp, iso-Glu, isohGlu, and isobhGlu. In some embodiments of Formula (I), A² is a D-amino acid, and A⁵ is either a D- or an L-amino acid residue. In some embodiments of Formula (II) A⁵ is a D-amino acid residue, particularly when A⁵ is Asp, Glu, hGlu or bhGlu. A³ and A⁴ can be D- or L-amino acid residues.

In both Formula (I) and Formula (II), A⁶ is (a) a hydroxy-substituted amino acid residue (HO-AA) which can be a D- or L-amino acid; (b) a C-terminal amide of the first HO-AA; (c) an oligopeptide comprising 2 to 5 amino acid residues wherein at least one residue thereof is the a HO-AA; or (d) a C-terminal amide of the oligopeptide; and the other residues of the oligopeptide comprise one or more residues selected from a D-amino acid residue, a L-amino acid residue, Gly, and a glycosylated second HO-AA; Carb is a carbohydrate group that is bonded to the sidechain oxygen of the HO-AA by a β-glycosidic bond; the sidechain amino group of A₂ is bonded to the sidechain carboxyl group of A⁵ when A⁵ is Asp, Glu, hGlu or bhGlu; and the sidechain amino group of A² is bonded to the α-carboxyl group of A⁵ when A⁵ is isoAsp, isoGlu, isohGlu, or isobhGlu. Unless otherwise specified, the glycoside sugars are D-sugars. Optionally, the C-terminus of A⁶ is amidated, e.g., as a primary amide.

In some embodiments of the compounds of Formulas (I) and (II), A¹ is L-Tyr.

In some embodiments of the compounds of Formulas (I) and (II), A³ is L-Trp, in which case the peptides are EM1 analogs, while in other embodiments, A³ is L-Phe, in which case the peptides are EM2 analogs.

In some embodiments of the compounds of Formulas (I) and (II), A⁴ is L-Phe.

In some embodiments of the compounds of Formulas (I) and (II), A¹ is L-Tyr, A³ is L-Trp, and A⁴ is L-Phe; while in other embodiments, A¹ is L-Tyr, A³ is L-Phe, and A⁴ is L-Phe.

In some embodiments of the compounds of Formula (II), A⁵ is Asp or Glu, preferably D-Asp or D-Glu.

Non-limiting examples of HO-AA residues include, e.g., a HO-AA such as Ser, Thr, homoSer, allo-Thr, cis-4-hydroxy-Pro, trans-4-hydroxy-Pro, cis-3-hydroxy-Pro, trans-3-hydroxy-Pro, and a C-terminal amide of any of the foregoing HOAA residues. The HO-AA can be in the L or D amino acid configuration.

As used herein, the notations “—NH₂”, “—NH₂”, and “CONH2” at the C-terminal end of a peptide interchangeably refer to amidation of the C-terminal carboxyl group as a primary amide (i.e., —(C=O)NH₂).

The sidechain oxygen of the HO-AA of A⁶ is glycosylated with a carbohydrate (Carb) group by a β-glycosidic bond between the carbohydrate and hydroxyl group. Non-limiting examples of Carb groups include monosaccharides, e.g., D-glucose (D-Glc), and disaccharides, e.g., D-lactose (D-Glc-β-D-Gal), ß-maltose, ß-lactose, ß-melibiose, trisaccharides, e.g., ß-maltotriose, both linear and branched and more complex carbohydrate moieties. Other examples include sucrose, trehalose, saccharose, maltose, cellobiose, gentibiose, isomaltose, and primeveose. Other glycosyl groups include galactose, xylose, mannose, manosaminic acid, fucose, GalNAc, GlcNAc, idose, iduronic acid, glucuronic acid and sialic acid. Unless otherwise specified, carbohydrates in Formula (I) and (II) are D-carbohydrates.

Oligopeptide A⁶ groups can include any amino acid residues in the oligopeptide chain in addition to the HO-AA. In some embodiments in which A⁶ is an oligopeptide, the oligopeptide comprises amino acids classified as “small amino acids” (see Betts and Russell p. 299) such as Gly, Ala, Cys and Pro to avoid steric interference at the opioid receptor active sites. Non-limiting examples of oligopeptide A⁶ groups with small amino acid residues include e.g., Gly-Ser, Gly-Ser-NH₂, Gly-Thr, Gly-Thr-NH₂, Ala-Ser, Ala-Ser-NH₂, Ala-Thr, Ala-Thr-NH₂, Pro-Ser, Pro-Ser-NH₂, Pro-Thr, Pro-Thr-NH₂, Gly-Gly-Ser-NH₂, Gly-Gly-Thr-NH₂, Gly-Gly-Pro-NH₂, Xaa-Xaa-Xaa-Ser-NH₂, Xaa-Xaa-Xaa-hydroxy-Pro-NH₂, Xaa-Xaa-Xaa-Xaa-Ser-NH₂, Xaa-Xaa-Xaa-Xaa-hydroxy-Pro-NH₂, and the like, in which each Xaa independently can be, e.g., Gly, Ala, Cys or Pro. In addition, oligopeptide A⁶ group can include more than one HO-AA, and optionally each HO-AA can be glycosylated.

Formula (III) provides an alternative structural representation of the compounds encompassed by Formula (I) in which A² is a D-amino acid residue; A³ is L-Trp or L-N-Me-Trp; and A⁴ is L-Phe, L-N-Me-Phe, L-p-X-Phe, and L-N-Me-p-X-Phe, wherein X is F, Cl, Br, or NO₂; as described herein, in order to emphasize the size of the ring portion and relative size of the sidechains:

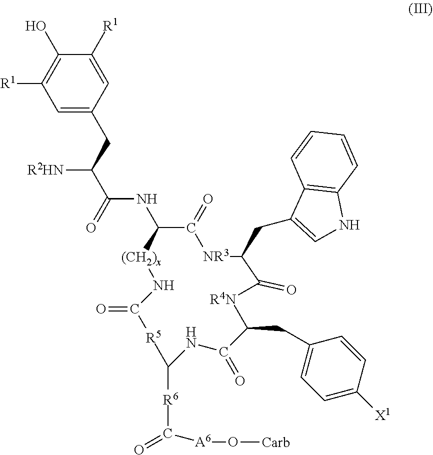

wherein
R¹, R², R³, and R⁴ are H or CH₃ (preferably R¹, R², R³, and R⁴ are H);
R⁵ is a covalent bond or —(CH₂)_{y}— (preferably R⁵ is a covalent bond);
R⁶ is a covalent bond or —(CH₂)_{z}— (preferably R⁶ is —(CH₂)_{z}—);
X¹ is H, F, Cl, Br, or NO₂ (preferably X¹ is H);
x is 1, 2, 3, 4, 5, or 6;
y is 1, 2, 3, or 4;
z is 1, 2, 3, or 4;
when R⁵ is a covalent bond, R⁶ is —(CH₂)_{z}—;
when R⁶ is a covalent bond, R⁵ is —(CH₂)_{y}—;
A⁶ is a D or L HO-AA selected from the group consisting of Ser, D-Ser, Thr, D-Thr, allo-Thr, and D-allo-Thr, homoSer, cis-4-hydroxy-Pro, trans-4-hydroxy-Pro, cis-3-hydroxy-Pro, trans-3-hydroxy-Pro; or A⁶ is an oligopeptide comprising 2 to 5 amino acid residues, at least one residue of the oligopeptide being a D or L HO-AA selected from the group consisting of Ser, D-Ser, Thr, D-Thr, allo-Thr, and D-allo-Thr, homoSer, cis-4-hydroxy-Pro, trans-4-hydroxy-Pro, cis-3-hydroxy-Pro, trans-3-hydroxy-Pro; Carb is a carbohydrate group that is bonded to the sidechain oxygen of the HO-AA by a β-D-glycosidic bond; and
the C-terminus of A⁶ optionally is a primary amide.

In some preferred embodiments of Formula (III), the C-terminus of A⁶ is a primary amide.

In some preferred embodiments of Formula (III), A⁶ is Ser, and Carb is D-glucose or D-lactose.

In some preferred embodiments of Formula (III), A⁶ is Ser-Ser, and the side-chain oxygens of each Ser is bound to a Carb (preferably D-glucose or D-lactose).

The compounds of Formula (II) in which A³ is L-Trp or L-N-Me-Trp, and A⁴ is L-Phe, L-N-Me-Phe, L-p-X-Phe, and L-N-Me-p-X-Phe, wherein X is F, Cl, Br, or NO₂, can be represented structurally as Formula (IV), again to illustrate the ring size variation:

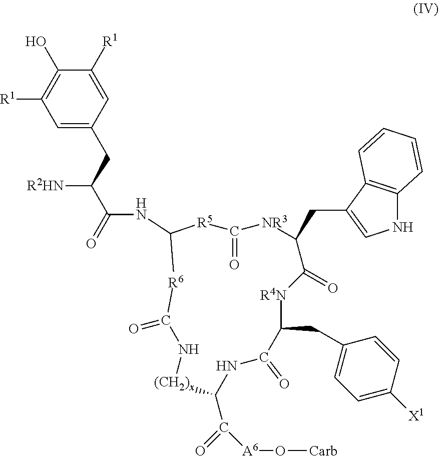

wherein
R¹, R², R³, and R⁴ are H or CH₃ (preferably R¹, R², R³, and R⁴ are H);
R⁵ is a covalent bond or —(CH₂)_{y}— (preferably R⁵ is a covalent bond);
R⁶ is a covalent bond or —(CH₂)_{z}— (preferably R⁶ is —(CH₂)_{z}—);
X¹ is H, F, Cl, Br, or NO₂ (preferably X¹ is H);
x is 1, 2, 3, 4, 5, or 6;
y is 1, 2, 3, or 4;
z is 1, 2, 3, or 4;
when R⁵ is a covalent bond, R⁶ is —(CH₂)_{z}—;
when R⁶ is a covalent bond, R⁵ is —(CH₂)_{y}—;
A⁶ is a D or L HO-AA selected from the group consisting of Ser, D-Ser, Thr, D-Thr, allo-Thr, and D-allo-Thr, homoSer, cis-4-hydroxy-Pro, trans-4-hydroxy-Pro, cis-3-hydroxy-Pro, trans-3-hydroxy-Pro; or A⁶ is an oligopeptide comprising 2 to 5 amino acid residues, at least one residue of the oligopeptide being a D or L HO-AA selected from the group consisting of Ser, D-Ser, Thr, D-Thr, allo-Thr, and D-allo-Thr, homoSer, cis-4-hydroxy-Pro, trans-4-hydroxy-Pro, cis-3-hydroxy-Pro, trans-3-hydroxy-Pro; Carb is a carbohydrate group that is bonded to the sidechain oxygen of the HO-AA by a β-D-glycosidic bond; and the C-terminus of A⁶ optionally is a primary amide.

In some preferred embodiments of Formula (IV), the C-terminus of A⁶ is a primary amide.

In some preferred embodiments of Formula (IV), A⁶ is Ser, and Carb is D-glucose or D-lactose.

In some preferred embodiments of Formula (IV), A⁶ is Ser-Ser, and the side-chain oxygens of each Ser is bound to a Carb (preferably D-glucose or D-lactose).

Table 1 illustrates the variation in ring size for various combinations of A² and A⁵ in Formula (I). Table 2 illustrates the variation in ring size for various combinations of A² and A⁵ in Formula (II). The ability to tailor the ring size of the peptides, along with the ability to select the relative positions of A² and A⁵ (compare Formula (I) with Formula (II)) can be advantageous for optimizing or tailoring different characteristics of the glycosylated peptides, such as, e.g., solubility in vehicle, blood stability, stomach acid stability, intestinal stability, storage stability, opioid receptor affinity, opioid receptor selectivity, and the like.

The ring size of the cyclic peptide portion of the compounds of Formula (I) can vary from 13 atoms to 22 atoms. This is evident from examination of the chemical structure of Formula (III): when R⁵ in is a covalent bond and x is 1 (i.e., when A² is Dpr and A⁵ is isoAsp, isoGlu, isohGlu, or isobhGlu in Formula (I)) there are 13 atoms making up the ring; whereas, when R⁵ is —(CH₂)_{y}—, x is 6, and y is 4 (i.e., when A² is bhLys and A⁵ is bhGlu in Formula (I)) there are 22 atoms in the ring. As in the case of Formula (I), the ring size in Formula (II) also varies depending on which A² and A⁵ residues are included in the material, although the variation follows a different pattern for the amino acid selections. The smallest ring size for compounds of Formula (II), 14 atoms, is obtained with A² being Dpr and A⁵ being Asp or isoAsp. The largest ring size (22 atoms) for compounds of Formula (II) is obtained with A² being bhLys and A⁵ being bhGlu or isobhGlu. As is evident in Tables 1 and 2, the same ring size can be obtained with a number of different choices for A² and A⁵, providing considerable flexibility in the ability to tailor the structure of the compounds in order to optimize and/or vary the physical, chemical, and biological properties of the compounds.

Additionally, the relative position of A² and A⁵ in the ring, along with the differences in ring size available by the selection of the A² and A⁵ residues, can have an effect on receptor binding affinity, binding strength, binding selectivity, and/or physical properties such as crystallinity, solubility, and solution stability. This provides many options for tailoring and optimizing activity, as well as the physical properties.

TABLE 1
Ring size for Compounds of Formula (I).

| Residue A² | Residue A⁵ | Ring Size (atoms) |
|---|---|---|
| Dpr | isoAsp, isoGlu, isohGlu, isobhGlu | 13 |
| Dpr | Asp | 14 |
| Dpr | Glu | 15 |
| Dpr | hGlu | 16 |
| Dpr | bhGlu | 17 |
| Dab | IsoAsp, isoGlu, isohGlu, isobhGlu | 14 |
| Dab | Asp | 15 |
| Dab | Glu | 16 |
| Dab | hGlu | 17 |
| Dab | bhGlu | 18 |
| Orn | isoAsp, isoGlu, isohGlu, isobhGlu | 15 |
| Orn | Asp | 16 |
| Orn | Glu | 17 |
| Orn | hGlu | 18 |
| Orn | bhGlu | 19 |
| Lys | isoAsp, isoGlu, isohGlu, isobhGlu | 16 |
| Lys | Asp | 17 |
| Lys | Glu | 18 |
| Lys | hGlu | 19 |
| Lys | bhGlu | 20 |
| hLys | isoAsp, isoGlu, isohGlu, isobhGlu | 17 |
| hLys | Asp | 18 |
| hLys | Glu | 19 |
| hLys | hGlu | 20 |
| hLys | bhGlu | 21 |
| bhLys | isoAsp, isoGlu, isohGlu, isobhGlu | 18 |
| bhLys | Asp | 19 |
| bhLys | Glu | 20 |
| bhLys | hGlu | 21 |
| bhLys | bhGlu | 22 |

TABLE 2
Ring size for Compounds of Formula (II).

| Residue A² | Residue A⁵ | Ring Size (atoms) |
|---|---|---|
| Dpr | Asp, isoAsp | 14 |
| Dpr | Glu, isoGlu | 15 |
| Dpr | hGlu, isohGlu | 16 |
| Dpr | bhGlu, isobhGlu | 17 |
| Dab | Asp, isoAsp | 15 |
| Dab | Glu, isoGlu | 16 |
| Dab | hGlu, isohGlu | 17 |
| Dab | bhGlu, isobhGlu | 18 |
| Orn | Asp, isoAsp | 16 |
| Orn | Glu, isoGlu | 17 |
| Orn | hGlu, isohGlu | 18 |
| Orn | bhGlu, isobhGlu | 19 |
| Lys | Asp, isoAsp | 17 |
| Lys | Glu, isoGlu | 18 |
| Lys | hGlu, isohGlu | 19 |
| Lys | bhGlu, isobhGlu | 20 |
| hLys | Asp, isoAsp | 18 |
| hLys | Glu, isoGlu | 19 |
| hLys | hGlu, isohGlu | 20 |
| hLys | bhGlu, isobhGlu | 21 |
| bhLys | Asp, isoAsp | 19 |
| bhLys | Glu, isoGlu | 20 |
| bhLys | hGlu, isohGlu | 21 |
| bhLys | bhGlu, isobhGlu | 22 |

As illustrated in Formula (III), when R⁵ is a covalent bond, then R⁶ is a methylene chain ranging from —CH₂— to —CH₂—CH₂—CH₂—CH₂, so that residue A⁶ is separated from the cyclic peptide ring by one to four carbon atoms relative to the case when R⁶ is a covalent bond. When A⁶ is an oligopeptide, the spacing of the cyclic peptide ring from the HO-AA of A⁶ increases by three atoms for each additional residue in the oligopeptide chain. Thus, the distance between the peptide ring and the HO-AA can be varied significantly, which may be advantageous, e.g., to optimize various characteristics of the glycosylated peptides, such as, e.g., solubility in vehicle, blood stability, stomach acid stability, intestinal stability, storage stability, opioid receptor affinity, opioid receptor selectivity, and the like.

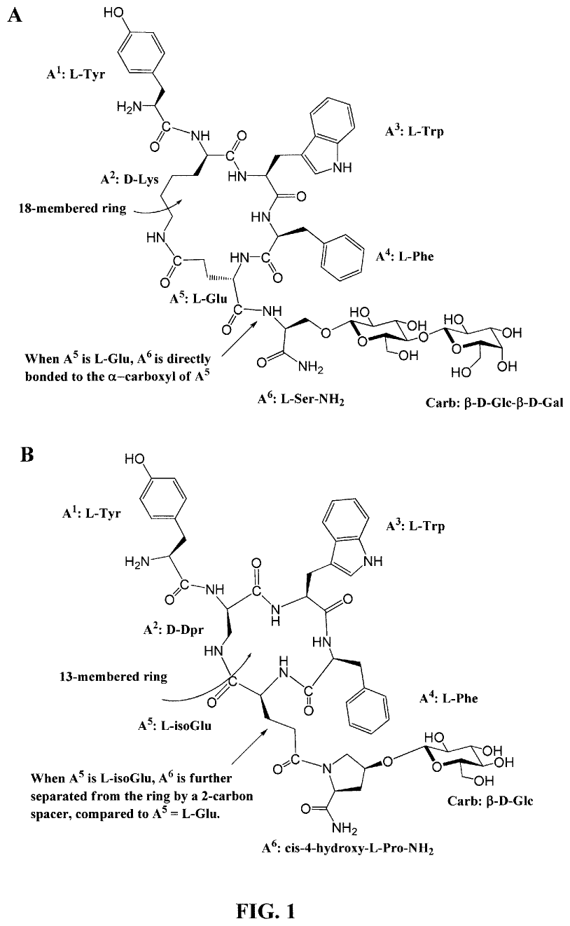

FIG. 1 Illustrates the chemical structures of two glycosylated cyclic EM1 analogs of Formula (I) with different peptide ring sizes. Panel A shows Analog A²: Tyr-cyclo[D-Lys-Trp-Phe-Glu]-Ser-NH₂ (SEQ ID NO: 1) glycosylated with β-D-Glc-μ-D-Gal (also known as β-D-Lact), having an 18-membered cyclic peptide ring, and Panel B shows Tyr-cyclo[D-Dpr-Trp-Phe-isoGlu]-(β-D-Glc)cis-4-hydroxy-L-Pro-NH₂ (SEQ ID NO: 2), having a 13-membered cyclic peptide ring.

### Synthesis of Glycosylated Cyclic EM1 Analogs

The glycosylated peptides of Formula (I) and (II) can be prepared by conventional solution phase or solid phase methods with the use of proper protecting groups and coupling agents. Such methods generally utilize various protecting groups on the various amino acid residues of the peptides. A suitable deprotection method is employed to remove specified (or all) protecting groups, including splitting off the resin if solid phase synthesis is applied. Apostol et al. (Peptides, 131, 2020, 170369) have reviewed peptide glycosylation. The peptides can be synthesized, for example, as described below.

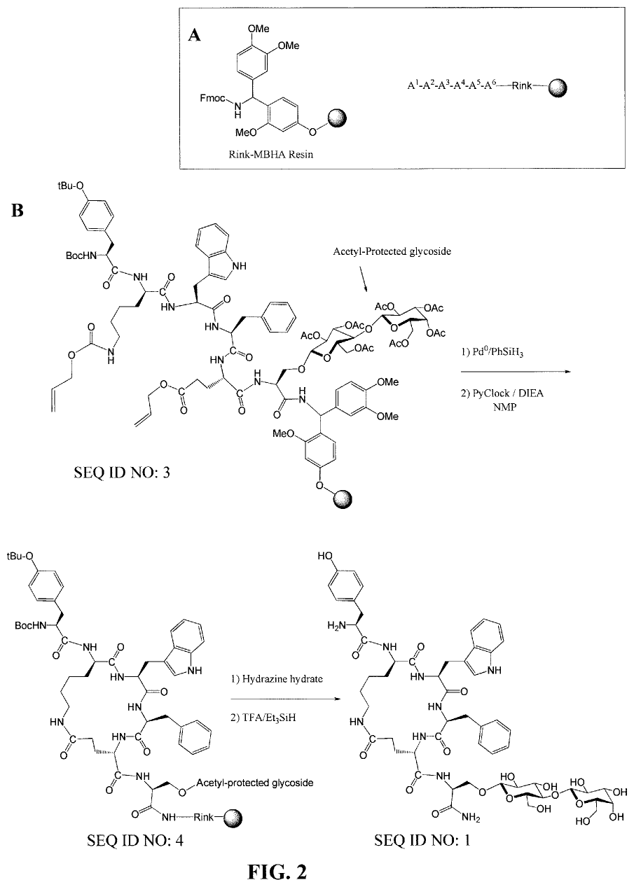

Peptides of Formula (I) and Formula (II) are synthesized on 4-methylbenzhydrylamine hydrochloride, polymer-bound-Rink (MBHA-Rink) resin via Fmoc-based solid-phase peptide synthesis (SPPS) starting with an acetate-protected glycosylated A⁶ residue directly bound to the MBHA-Rink resin, and then the remaining residues are added sequentially in the order A⁵, A⁴, A³, A² and A¹ (see e.g., FIG. 2, panel A) for compounds of Formula (I), and in the order A², A⁴, A³, A⁵ and A¹ for Formula (II) compounds. A t-butyl ether group is included for A¹ sidechain protection and Boc (t-butyloxycarbonyl) is used for A¹ amino protection. The peptide is assembled on a MBHA-Rink resin by repetitive removal of the Fmoc protecting group and coupling of protected amino acid. For example, HBTU (O-benzotriazole-N,N,N′,N′-tetramethyluronium hexafluorophosphate; CAS #94790-37-1) and HOBT (N-hydroxybenzotriazole; CAS #2592-95-2) are used as coupling reagents in N,N-dimethylformamide (DMF) and diisopropylethylamine (DIPEA) is used as a base. The A² and A⁵ amino and carboxyl groups that will eventually be coupled to form the peptide ring structure are orthogonally protected (Isidro-Llobet, Alvarez, and Albericio 2009) as follows: the amino sidechain of A² (i.e., D-Lysine, D-Orn, D-Dab or D-Dpr) is protected by an allyloxycarbonyl (Alloc) group, while the sidechain carboxyl of A⁵ is protected as an allyl (All) ester when A⁵ is Asp, Glu, hGlu or bhGlu; or the α carboxyl group of A⁵ is protected as the allyl ester when A⁵ is isoAsp, isoGlu, isohGlu or isobhGlu (see e.g., FIG. 2, panel B). These two protecting groups can be removed simultaneously by hydrogen atom transfer (e.g., with Pd⁰ and phenylsilane, PhSiH₃), and ring closure effected with a peptide condensation reagent, such as 6-chloro-benzotriazole-1-yloxy-tris-pyrrolidinophosphonium hexafluorophosphate (PyClock), to produce a cyclic peptide ring. The resulting peptide is deprotected with hydrazine hydrate to remove the acetate groups from the sugar, followed by trifluoroacetic acid (TFA and triethylsilane, Et₃SiH) to remove the remaining sidechain protection and cleave the completed glycopeptide from the resin.

Crude peptide is precipitated with diethyl ether and collected by filtration. The peptide can be purified by high performance liquid chromatography or other methods of peptide purification known in the peptide synthesis art. For example, the solids obtained above can be dissolved in DMF or a similar polar aprotic solvent. The mixture is stirred at room temperature for about 1 hour. Solvent is removed in vacuo. The residual oil is dissolved in 10% aqueous acetonitrile (MeCN/H₂O) and lyophilized to obtain the peptides in solid form. Purification of the crude lyophilized peptides is performed with reverse-phase high performance liquid chromatography (RP-HPLC) by techniques that are well known in the peptide and protein arts.

### Pharmaceutical Preparations.

Also described herein are pharmaceutical preparations that contain a pharmaceutically effective amount of the glycosylated peptides in a pharmaceutically acceptable carrier (e.g., a diluent, complexing agent, additive, excipient, adjuvant and the like). The glycosylated peptide can be present for example in a salt form, a micro-crystal form, a nano-crystal form, a co-crystal form, a nanoparticle form, a microparticle form, or an amphiphilic form. Salt forms can be, e.g., salts of inorganic acids such as hydrochloride salts, phosphate salts, sulfate salts, bisulfate salts, hemisulfate salts, and the like; or salts of organic acids, such as acetate salts, aspartate salts, citrate salts, fumarate salts, maleate salts, malate salts, lactate salts, hippurate salts, tartrate salts, gluconate salts, succinate salts, and the like.

The carrier can be an organic or inorganic carrier, or a combination thereof, which is suitable for external, enteral or parenteral applications. The glycosylated peptides can be compounded, for example, with the usual non-toxic, pharmaceutically acceptable carriers for tablets, pellets, capsules, liposomes, suppositories, intranasal sprays, solutions, emulsions, suspensions, aerosols, targeted chemical delivery systems, and any other form suitable for use. Non-limiting examples of carriers that can be used include water, glucose, lactose, gum acacia, gelatin, mannitol, starch paste, magnesium trisilicate, talc, corn starch, keratin, colloidal silica, potato starch, urea and other carriers suitable for use in manufacturing preparations, in solid, semisolid, liquid or aerosol form. In addition, auxiliary agents, stabilizing agents, thickening agent, coloring agents, and perfumes can be used in the compositions.

In another aspect, pharmaceutical compositions useful for treating pain and related conditions utilizing the glycosylated peptide compounds of Formula (I) and Formula (II) are described herein. The pharmaceutical compositions comprise at least one glycosylated peptide of Formula (I) and/or Formula (II) in combination with a pharmaceutically acceptable carrier, vehicle, or diluent, such as an aqueous buffer at a physiologically acceptable pH (e.g., pH 7 to 8.5), a polymer-based nanoparticle vehicle, a liposome, and the like. The pharmaceutical compositions can be delivered in any suitable dosage form, such as a liquid, gel, solid, cream, or paste dosage form. In one embodiment, the compositions can be adapted to give sustained release of the peptide.

In some embodiments, the pharmaceutical compositions include, but are not limited to, those forms suitable for oral, rectal, nasal, inhalant, topical, (including buccal and sublingual), transdermal, vaginal, parenteral (including intramuscular, subcutaneous, and intravenous), spinal (epidural, intrathecal), and central (intracerebroventricular) administration. The compositions can, where appropriate, be conveniently provided in discrete dosage units. The pharmaceutical compositions can be prepared by any of the methods well known in the pharmaceutical arts. Some preferred modes of administration include intravenous (iv), topical, subcutaneous, oral and spinal.

Pharmaceutical compositions suitable for oral administration include capsules, cachets, or tablets, each containing a predetermined amount of one or more of the peptides, as a powder or granules. In another embodiment, the oral composition is a solution, a suspension, or an emulsion. Alternatively, the peptides can be provided as a bolus, electuary, or paste. Tablets and capsules for oral administration can contain conventional excipients such as binding agents, fillers, lubricants, disintegrants, colorants, flavoring agents, preservatives, or wetting agents. The tablets can be coated according to methods well known in the art, if desired. Oral liquid preparations include, for example, aqueous or oily suspensions, solutions, emulsions, syrups, or elixirs. Alternatively, the compositions can be provided as a dry product for constitution with water or another suitable vehicle before use. Such liquid preparations can contain conventional additives such as suspending agents, emulsifying agents, non-aqueous vehicles (which may include edible oils), preservatives, and the like. The additives, excipients, and the like typically will be included in the compositions for oral administration within a range of concentrations suitable for their intended use or function in the composition, and which are well known in the pharmaceutical formulation art. The glycosylated peptides of Formula (I) and/or Formula (II) will be included in the compositions within a therapeutically useful and effective concentration range, as determined by routine methods that are well known in the medical and pharmaceutical arts. For example, a typical composition can include one or more of the peptides at a concentration in the range of at least about 0.01 nanomolar to about 1 molar, preferably at least about 1 nanomolar to about 100 millimolar.

Pharmaceutical compositions for parenteral, spinal, or central administration (e.g. by bolus injection or continuous infusion) or injection into amniotic fluid can be provided in unit dose form in ampoules, pre-filled syringes, small volume infusion, or in multi-dose containers, and preferably include an added preservative. The compositions for parenteral administration can be suspensions, solutions, or emulsions, and can contain excipients such as suspending agents, stabilizing agents, and dispersing agents. Alternatively, the glycosylated peptides of Formula (I) and/or Formula (II) can be provided in powder form, obtained by aseptic isolation of sterile solid or by lyophilization from solution, for constitution with a suitable vehicle, e.g. sterile, pyrogen-free water, before use. The additives, excipients, and the like typically will be included in the compositions for parenteral administration within a range of concentrations suitable for their intended use or function in the composition, and which are well known in the pharmaceutical formulation art. The glycosylated peptides of Formula (I) and/or Formula (II) will be included in the compositions within a therapeutically useful and effective concentration range, as determined by routine methods that are well known in the medical and pharmaceutical arts. For example, a typical composition can include one or more of the peptides at a concentration in the range of at least about 0.01 nanomolar to about 100 millimolar, preferably at least about 1 nanomolar to about 10 millimolar.

Pharmaceutical compositions for topical administration of the glycosylated peptides of Formula (I) and/or Formula (II) to the epidermis (mucosal or cutaneous surfaces) can be formulated as ointments, creams, lotions, gels, or as a transdermal patch. Such transdermal patches can contain penetration enhancers such as linalool, carvacrol, thymol, citral, menthol, t-anethole, and the like. Ointments and creams can, for example, include an aqueous or oily base with the addition of suitable thickening agents, gelling agents, colorants, and the like. Lotions and creams can include an aqueous or oily base and typically also contain one or more emulsifying agents, stabilizing agents, dispersing agents, suspending agents, thickening agents, coloring agents, and the like. Gels preferably include an aqueous carrier base and include a gelling agent such as cross-linked polyacrylic acid polymer, a derivatized polysaccharide (e.g., carboxymethyl cellulose), and the like. The additives, excipients, and the like typically will be included in the compositions for topical administration to the epidermis within a range of concentrations suitable for their intended use or function in the composition, and which are well known in the pharmaceutical formulation art. The glycosylated peptides of Formula (I) and/or Formula (II) will be included in the compositions within a therapeutically useful and effective concentration range, as determined by routine methods that are well known in the medical and pharmaceutical arts. For example, a typical composition can include one or more of the peptides at a concentration in the range of at least about 0.01 nanomolar to about 1 molar, preferably at least about 1 nanomolar to about 100 millimolar.

Pharmaceutical compositions suitable for topical administration in the mouth (e.g., buccal or sublingual administration) include lozenges comprising the peptide in a flavored base, such as sucrose, acacia, or tragacanth; pastilles comprising the peptide in an inert base such as gelatin and glycerin or sucrose and acacia; and mouthwashes comprising the active ingredient in a suitable liquid carrier. The pharmaceutical compositions for topical administration in the mouth can include penetration enhancing agents, if desired. The additives, excipients, and the like typically will be included in the compositions of topical oral administration within a range of concentrations suitable for their intended use or function in the composition, and which are well known in the pharmaceutical formulation art. The peptides of the present invention will be included in the compositions within a therapeutically useful and effective concentration range, as determined by routine methods that are well known in the medical and pharmaceutical arts. For example, a typical composition can include one or more of the peptides at a concentration in the range of at least about 0.01 nanomolar to about 1 molar, preferably at least about 1 nanomolar to about 100 millimolar.

A pharmaceutical composition suitable for rectal administration comprises a glycosylated peptide of Formula (I) and/or Formula (II) in combination with a solid or semisolid (e.g., cream or paste) carrier or vehicle. For example, such rectal compositions can be provided as unit dose suppositories. Suitable carriers or vehicles include cocoa butter and other materials commonly used in the art. The additives, excipients, and the like typically will be included in the compositions of rectal administration within a range of concentrations suitable for their intended use or function in the composition, and which are well known in the pharmaceutical formulation art. The glycosylated peptides of Formula (I) and/or Formula (II) will be included in the compositions within a therapeutically useful and effective concentration range, as determined by routine methods that are well known in the medical and pharmaceutical arts. For example, a typical composition can include one or more of the peptides at a concentration in the range of at least about 0.01 nanomolar to about 1 molar, preferably at least about 1 nanomolar to about 100 millimolar.

According to one embodiment, pharmaceutical compositions suitable for vaginal administration are provided as pessaries, tampons, creams, gels, pastes, foams, or sprays containing a peptide of the invention in combination with carriers as are known in the art. Alternatively, compositions suitable for vaginal administration can be delivered in a liquid or solid dosage form. The additives, excipients, and the like typically will be included in the compositions of vaginal administration within a range of concentrations suitable for their intended use or function in the composition, and which are well known in the pharmaceutical formulation art. The glycosylated peptides of Formula (I) and/or Formula (II) will be included in the compositions within a therapeutically useful and effective concentration range, as determined by routine methods that are well known in the medical and pharmaceutical arts. For example, a typical composition can include one or more of the peptides at a concentration in the range of at least about 0.01 nanomolar to about 1 molar, preferably at least about 1 nanomolar to about 100 millimolar.

Pharmaceutical compositions suitable for intra-nasal administration are also described herein. Such intra-nasal compositions comprise a glycosylated peptide of Formula (I) and/or Formula (II) in a vehicle and suitable administration device to deliver a liquid spray, dispersible powder, or drops. Drops may be formulated with an aqueous or non-aqueous base also comprising one or more dispersing agents, solubilizing agents, or suspending agents. Liquid sprays are conveniently delivered from a pressurized pack, an insufflator, a nebulizer, or other convenient means of delivering an aerosol comprising the peptide. Pressurized packs comprise a suitable propellant such as dichlorodifluoromethane, trichlorofluoromethane, dichlorotetrafluoroethane, carbon dioxide, or other suitable gas as is well known in the art. Aerosol dosages can be controlled by providing a valve to deliver a metered amount of the peptide. Alternatively, pharmaceutical compositions for administration by inhalation or insufflation can be provided in the form of a dry powder composition, for example, a powder mix of the peptide and a suitable powder base such as lactose or starch. Such powder composition can be provided in unit dosage form, for example, in capsules, cartridges, gelatin packs, or blister packs, from which the powder can be administered with the aid of an inhalator or insufflator. The additives, excipients, and the like typically will be included in the compositions of intra-nasal administration within a range of concentrations suitable for their intended use or function in the composition, and which are well known in the pharmaceutical formulation art. The glycosylated peptides of Formula (I) and/or Formula (II) will be included in the compositions within a therapeutically useful and effective concentration range, as determined by routine methods that are well known in the medical and pharmaceutical arts. For example, a typical composition can include one or more of the peptides at a concentration in the range of at least about 0.01 nanomolar to about 1 molar, preferably at least about 1 nanomolar to about 100 millimolar.

Optionally, the pharmaceutical compositions can include one or more other therapeutic agent, e.g., as a combination therapy. The additional therapeutic agent will be included in the compositions within a therapeutically useful and effective concentration range, as determined by routine methods that are well known in the medical and pharmaceutical arts. The concentration of any particular additional therapeutic agent may be in the same range as is typical for use of that agent as a monotherapy, or the concentration may be lower than a typical monotherapy concentration if there is a synergy when combined with a peptide of the present invention.

In another aspect, the present invention provides for the use of the glycosylated peptide of Formula (I) and/or Formula (II) for treatment of pain, treatment of discomfort associated with gastrointestinal disorders, treatment of drug dependence, and OUD. Methods for providing analgesia (alleviating or reducing pain), relief from gastrointestinal disorders such as diarrhea, and therapy for drug dependence and OUD in patients, such as mammals, including humans, comprise administering to a patient suffering from one of the aforementioned conditions an effective amount of a glycosylated peptide of Formula (I). Diarrhea may be caused by a number of sources, such as infectious disease, cholera, or an effect or side-effect of various drugs or therapies, including those used for cancer therapy. Preferably, the peptide is administered parenterally or enterally. The dosage of the effective amount of the peptides can vary depending upon the age and condition of each individual patient to be treated. However, suitable unit dosages typically range from about 0.01 to about 100 mg. For example, a unit dose can be in the range of about 0.2 mg to about 50 mg. Such a unit dose can be administered more than once a day, e.g., two or three times a day.

All of the embodiments of the glycosylated peptides of Formula (I) and Formula (II) can be in the “isolated” state. For example, an “isolated” peptide is one that has been completely or partially purified. In some instances, the isolated compound will be part of a greater composition, buffer system or reagent mix. Optionally, the isolated peptide may be in the form of a pharmaceutically acceptable salt (e.g., as a hydrochloride salt, a trifluoroacetate salt, a trifluoromethane sulfonic acid salt, and the like). In other circumstances, the isolated peptide may be purified to homogeneity. A composition may comprise the peptide or compound at a level of at least about 50, 80, 90, or 95% (on a molar basis or weight basis) of all the other species that are also present therein. Mixtures of the glycosylated peptides of Formula (I) and/or Formula (II) may be used in practicing methods described herein.

Also described herein are methods of using the peptides of Formula (I) and Formula (II) disclosed herein in medicinal formulations or as therapeutic agents, for example. These methods may involve the use of a single peptide, or multiple peptides in combination (i.e., a mixture). Accordingly, certain embodiments are drawn to medicaments comprising the glycosylated peptide of Formula (I) and/or Formula (II), and methods of manufacturing such medicaments.

As used herein, the terms “reducing,” “inhibiting,” “blocking,” “preventing”, alleviating,” or “relieving” when referring to a compound (e.g., a peptide), mean that the compound brings down the occurrence, severity, size, volume, or associated symptoms of a condition, event, or activity by at least about 7.5%, 10%, 12.5%, 15%, 17.5%, 20%, 22.5%, 25%, 27.5%, 30%, 35%, 40%, 45%, 50%, 55%, 60%, 65%, 70%, 75%, 80%, 90%, or 100% compared to how the condition, event, or activity would normally exist without application of the compound or a composition comprising the compound. The terms “increasing,” “elevating,” “enhancing,” “upregulating”, “improving,” or “activating” when referring to a compound mean that the compound increases the occurrence or activity of a condition, event, or activity by at least about 7.5%, 10%, 12.5%, 15%, 17.5%, 20%, 22.5%, 25%, 27.5%, 30%, 35%, 40%, 45%, 50%, 55%, 60%, 65%, 70%, 75%, 80%, 90%, 100%, 150%, 200%, 250%, 300%, 400%, 500%, 750%, or 1000% compared to how the condition, event, or activity would normally exist without application of the compound or a composition comprising the compound.

The following examples are included to illustrate certain aspects of the materials and methods described herein. It should be appreciated by those of skill in the art that the techniques disclosed in the examples, which represent techniques known to function well in producing or using the described glycosylated peptides, can be considered to constitute preferred materials and methods. However, those of skill in the art should, in light of the present disclosure, appreciate that many changes can be made in the specific disclosed embodiments and still obtain a like or similar result without departing from the spirit and scope of the inventions set forth in the appended claims. The examples are provided for illustration purposes only and are not intended to be limiting.

### EXAMPLES

### Examples 1. Peptide Synthesis

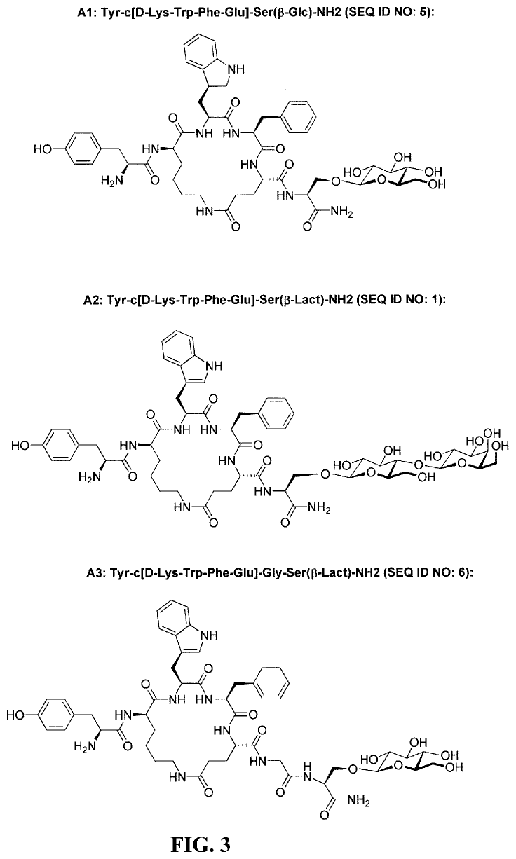

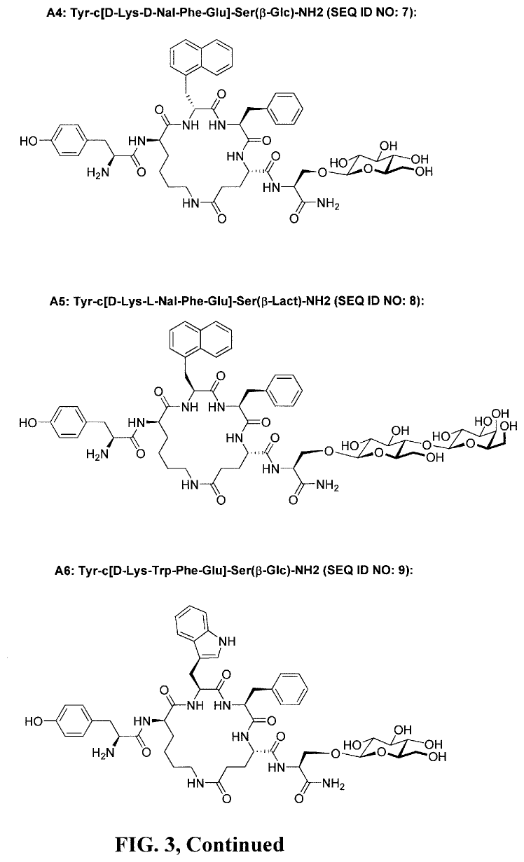

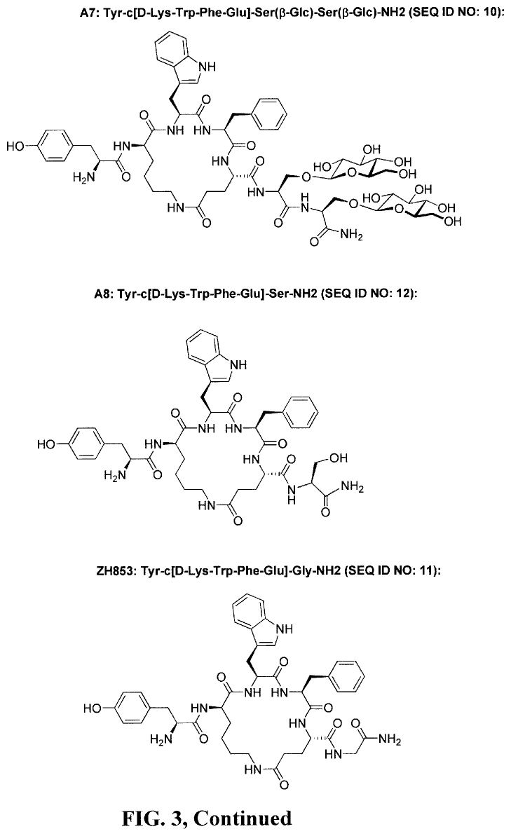

Chemicals/Peptide Synthesis: Morphine was generously supplied by the NIDA Drug Supply Program. ZH853 was custom synthesized and certified as >95% purity by Anaspec (Fremont, CA). Peptides A¹ to A⁸ were synthesized as described above by the laboratory of Robin Polt (see FIG. 2, FIG. 3 and Table 3).

TABLE 3
Analogs Tested:

| Analog | A¹ | A² | A³ | A⁴ | A⁵ | A⁶ | Carb |
|---|---|---|---|---|---|---|---|
| A1 | L-Tyr | D-Lys | L-Trp | -Phe | L-Glu | L-Ser | β-D-Glc |
| A2 | L-Tyr | D-Lys | L-Trp | -Phe | L-Glu | L-Ser | β-D-Lact |
| A3 | L-Tyr | D-Lys | L-Trp | -Phe | L-Glu | L-Ser | β-D-Glc |
| A4 | L-Tyr | D-Lys | 1-D-Nal | -Phe | L-Glu | L-Ser | β-D-Glc |
| A5 | L-Tyr | D-Lys | 1-D-Nal | -Phe | L-Glu | L-Ser | β-D-Lact |
| A6 | L-Tyr | D-Lys | 1-D-Nal | -Phe | L-Glu | L-Ser | β-D-Glc |
| A7 | L-Tyr | D-Lys | L-Trp | -Phe | L-Glu | L-Ser | β-D-Glc |
| A8 | L-Tyr | D-Lys | L-Trp | -Phe | L-Glu | L-Ser | none |

FIG. 2 schematically illustrates aspects of the synthesis of Analog A2: Tyr-cyclo[D-Lys-Trp-Phe-Glu]-(β-Lact)Ser-NH₂ (SEQ ID NO: 1). Panel A illustrates the functional portion of MBHA-Rink resin and the order in which the residues of the peptide are added to the resin-bound peptide of Formula (I). Panel B illustrates the ring closure of the cyclic peptide A2, as well as deprotection and cleavage of the peptide from the resin.

The unglycosylated cyclic peptide ZH853 was custom synthesized as the acetate salt and certified as >95% purity by Anaspec (Fremont, CA). Glycopeptides A1-A7 and the unglycosylated control peptide A8 were synthesized as described below, with an estimated >95% purity by HPLC.

O-Linked glycopeptides were synthesized using well-established solid phase methods based on FMOC protection using an automated PRELUDE peptide synthesizer (Gyros-PTI) or manually with a simple fritted syringe (Isidro-Llobet et al., 2009).

The first amino acid on the resin, e.g., either Fmoc-Ser[O-β-D-Glc(OAc)₄]—OH or Fmoc-Ser[O-β-Lact(OAc)₇]—OH, was manually coupled to MBHA-Rink amide resin (200 mesh, 0.83 mmol/g original loading capacity) 0.8 eq. of the Fmoc-amino acids and capping with Ac₂O to provide a final resin loading of 0.5 to 0.7 mmol/g of Fmoc-amino acid.

For each gram of MBHA-Rink amide resin (200 mesh, 0.83 mmol/gram), 0.8 mmol of amino acid was used. Using a fritted syringe as the reaction vessel, the resin was swelled in DMF for 10 minutes, then washed twice with dimethylformamide (DMF) for 2 minutes each. Fmoc removal was accomplished using 2% DBU plus 2% piperidine in DMF. After the treatment, the resin was washed six times with DMF for 2 minutes each. For the Fmoc glyco-amino acid activation, chlorohydroxybenzotriazole (Cl-HOBt) with diisopropylcarbodiimide (DIC) was used. Loading of the following Fmoc-Amino acids (3 eq./resin mmol) were performed with N,N,N′,N′-Tetramethyl-O-(1H-benzotriazol-1-yl)uronium hexafluoro-phosphate, 0-(Benzotriazol-1-yl)-N,N,N′,N′-tetramethyluronium hexafluorophosphate (HBTU, (3 equiv/resin mmol), and N-methyl morpholine (12 eq./resin mmol) using the PRELUDE peptide synthesizer.

At the cyclisation site, Fmoc-Glu(All) and Fmoc Lys(Alloc) were used to provide selective deprotection (Thieret et al., 1997). After the full peptide was synthesized on the resin, the All and Alloc protecting groups were removed by using 0.1 eq./resin mmol of Pd(PPh₃)₄ with the presence of 20 eq./resin mmol of PhSiH₃ in dichloromethane (DCM), following by washing six times with DCM for 2 minutes each, and then washing four times with DMF for 2 minutes.

The lactam ring was formed by 4 eq./resin mmol of PyClock and 12 eq diisopropylethyl amine (DIEA) in NMP. Then the resin was washed four times with DMF for 2 minutes, and intensively washed twice with 0.5% sodium diethyldithiocarbamate (DEDTC) for 4 min in DMF, followed by six times with DMF for 2 minutes each, as well as six times with DCM for 2 minutes each. The resin was dried in vacuum.

Acetyl groups were removed from the sugar moieties with 50% hydrazine hydrate and in NMP (H₂NNH₂·H₂O/NMP) for 1 hour with gentle mixing. The solution was expelled and the resin was recharged with the H₂NNH₂·H₂O/NMP solution and gently mixed for an additional 3 hours, washed with six times with DMF for 2 minutes, six times with DCM for 2 minutes, and dried in vacuo.

A cleavage cocktail containing trifluoroacetic acid (TFA), DCM, H₂O, with scavengers triethylsilane (Et₃SiH) and anisole (9:1:0.2:0.3:0.05), was used to simultaneously remove the amino acid sidechain and N-terminus protecting groups (t-butyl ether and Boc protecting groups, respectively) and cleave the cyclic glycopeptide from the resin. The resin was treated with 1 mL cleavage cocktail per 100 mg resin (based on initial mass) and tumbled for 1 hour. The cleavage cocktail solution was collected, the volume reduced under argon to about 1/3 the original volume. Addition of diethyl ether (Et₂O) at 0° C. precipitated the crude glycopeptide, which was then centrifuged, and the Et₂O was poured away to obtain the crude peptide, which was then was dried vacuum. The resulting grayish solid was dissolved in H₂O, frozen, and lyophilized prior to purification by reverse phase high performance liquid chromatography (RP-HPLC) using a semi-preparative (250×25 mm) or preparative (250×55 mm)C-18 column employing a H₂O: CH₃CN gradient containing 0.1% trifluoroacetic acid (TFA). Purities of the products (typically 9-10% yield) were confirmed using analytical RP-HPLC (250×4.0 mm) and MS/MS with Collision Induced Decomposition (CID) and by high field (600 or 800 MHz) NMR.

### Example 2. Peptide Binding Studies

Receptor Binding Profiles and Ki: The receptor binding an Ki determinations were generously provided by the National Institute of Mental Health's Psychoactive Drug Screening Program (PDSP), Directed by Bryan L. Roth at the University of North Carolina at Chapel Hill and Project Officer Jamie Driscoll at NIMH, Bethesda MD, USA.

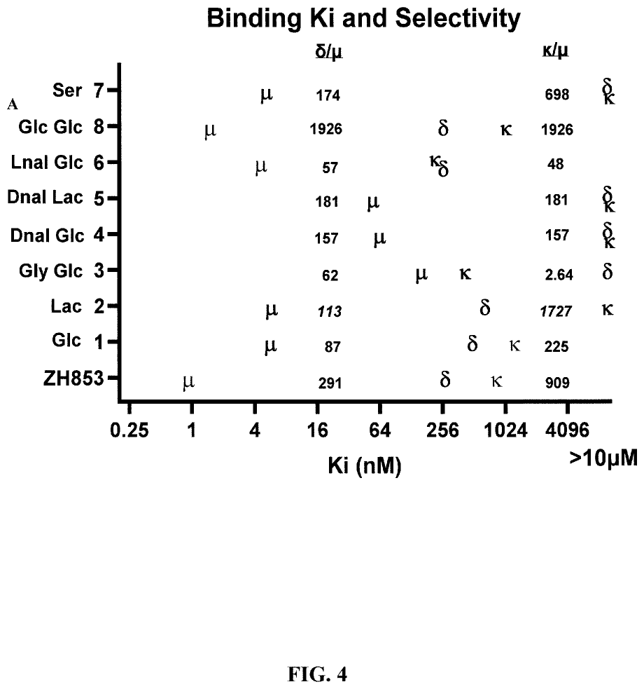

FIG. 4 graphically shows receptor binding affinity for mu, delta and kappa (μ, δ, κ, MOR, DOR, KOR) opioid receptors. Table 4 provides binding (Ki) values (in nM units; higher values indicate lower binding affinity) and log Ki, for the mu opioid receptor (MOR), the delta opioid receptor (DOR) and kappa opioid receptor (KOR), as well as ratios of DOR-to-MOR (delta/mu) and KOR-to-MOR binding, for glycosylated Analogs A1, A2, A3, A4, A5, A6 and A7 compared to unglycosylated A8 and ZH853.

The reference compound ZH853, with a subnanomolar Ki, showed the highest MOR affinity followed by the unglycosylated-Ser Analog A8 (identified as 8 in the figure). Glycosylation reduced the MOR affinity approximately 5-fold for analogs A1, A2, A6 and A7 (identified as 1, 2, 6, and 7, respectively, in FIG. 4). Substitution of Trp at position 3 with D-Nal (Analogs A4 and A5, identified as 4 and 5, respectively, in FIG. 4) further reduced MOR binding an additional 11-fold and eliminated delta and kappa binding. L-Nal substitution (A6, identified as 6 in FIG. 4), however, did not reduce MOR affinity but increased KOR affinity, resulting in the least selective compound. By contrast, a double glycoside (A7, identified as 7 in FIG. 4) did not change MOR affinity but reduced DOR and KOR to below detectable levels, resulting in the most selective MOR binding. Unexpectedly, insertion of a Gly spacer before the Ser+ glucoside (A3, identified as 3 in FIG. 4) reduced MOR and increased KOR affinity, resulting in the lowest MOR affinity and the only analog with higher KOR than DOR affinity. Binding studies also showed that the analogs did not bind significantly (>10 μM) to 28 key, off-target sites: 8 Adrenergic receptor cites, 7 Serotonin receptor sites, 4 Dopamine receptor sites, 3 Histamine receptor sites, 2 Muscarinic receptor sites, 2 Benzodiazapine sites, GABAa receptor, and Sigma-2 receptor, as well as the Dopamine Transporter (DAT) receptor.

TABLE 4

| | | | | Ki (nM) | | | Log Ki | | |
|---|---|---|---|---|---|---|---|---|---|
| Entry | C-Terminus | del/mu | kap/mu | MOR | DOR | KOR | MOR | DOR | KOR |
| ZH853 | Gly-CONH2 | 291 | 909 | 0.93 | 271 | 845 | −9.03 | −6.57 | −6.07 |
| A8 | Ser(OH)CONH2 | 174 | 698 | 1.51 | 263 | 1055 | −8.82 | | −5.98 |
| A7 | Ser(Glc)-Ser(Glc)CONH2 | 1926 | 1926 | 5.19 | 10,000 | 10,000 | −8.28 | | |
| A6 | Ser(Glc)CONH2 | 57 | 48 | 4.61 | 262 | 220 | −8.34 | −6.58 | −6.66 |
| A5 | Ser(Lact)CONH2 | 181 | 181 | 55.25 | 10,000 | 10,000 | −7.26 | | |
| A4 | Ser(Glc)CONH2 | 157 | 157 | 63.52 | 10,000 | 10,000 | −7.20 | | |
| A3 | Gly-Ser(Glc)CONH2 | 62 | 2.64 | 160.62 | 10,000 | 423.7 | −6.79 | | |
| A2 | Ser(Lact)CONH2 | 113 | 1727 | 5.79 | 655.7 | 10,000 | −8.24 | −6.18 | |
| A1 | Ser(Glc)CONH2 | 87 | 225 | 5.65 | 495.6 | 1272 | −8.25 | −6.30 | −5.90 |

### Example 3. In Vivo Evaluation of the Peptides

Animals: Male and female CD-1 and DBA mice (21-29 g weight at testing), were obtained from Charles River, (Wilmington, MA) and housed in a 12-h light/dark cycle. All experiments were approved by the Tulane Institutional Animal Care and Use Committee and conducted according to the NIH Guide for the Care and Use of Laboratory Animals. All efforts were made to minimize animal suffering, and to reduce the number of animals used.

Antinociception: Antinociception was determined in standard tail flick (TF) and hot plate (HP) tests described below.

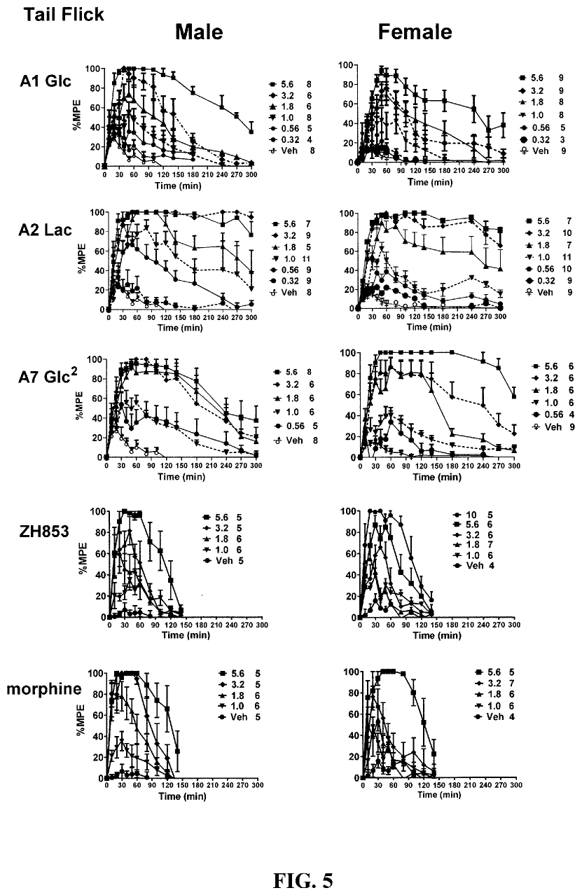

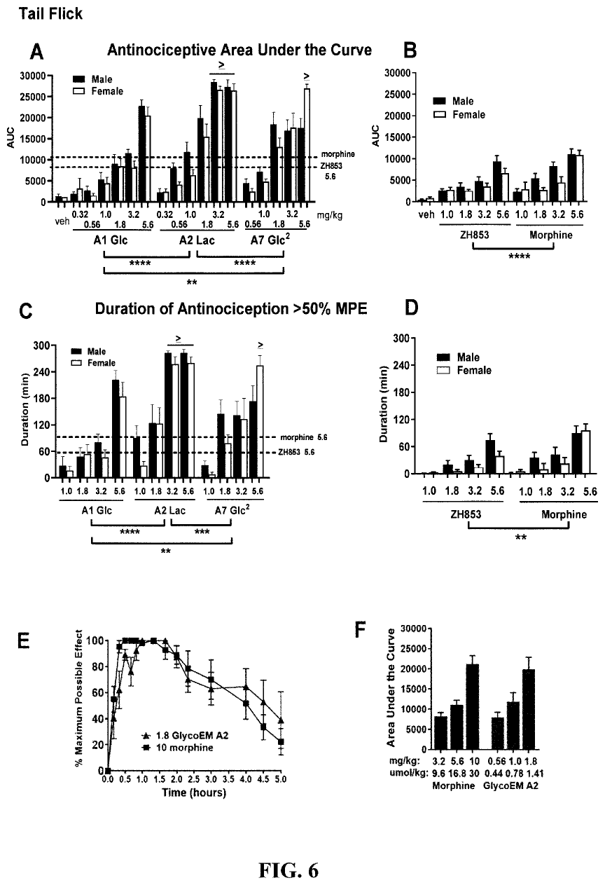

Tail Flick (TF): CD1 mice were used for this test. The latency to withdraw the tail from a heat source was automatically measured (IITC, Woodland Hills, CA). Baseline latencies were 3-4 s with a cutoff time of 9 s to prevent tissue damage. Percent Maximum Possible Effect (% MPE) was determined as [(latency-baseline latency)/(9-baseline latency)]*100. Peak responses were later for the glycopeptides (40-60 min) compared to the reference compound ZH853 (30-40 min) and morphine (20-30 min). The average MPE at each of those respective times was therefore calculated and used as the peak response for determining ED₅₀'s. Initial tests, based on previous studies with ZH853, were 3 hr in duration. As shown in FIG. 5, however, some of the glycopeptide analog doses, particularly Analog A2, produced an unexpectedly long duration of antinociception that remained well above 50% MPE at 3 hr. Additional subgroups (n=4-5) were therefore tested for 5 hr. For A7, all animals were tested for 5h. For the time course figure (FIG. 5) and the dose-response curve/ED₅₀ calculations (FIG. 6), which were based on ≤1 hr values, all animals were used. Duration of antinociception was assessed using both the area under the curve (AUC) and duration of antinociception, calculated as the time from the first to the last time point greater than or equal to 50% MPE. For these two measures, because a majority of animals in the top 2 doses for A2, and the top dose of A1, were greater than 50% MPE at 3 hr, therefore underestimating the true AUC and duration, only the 5h subgroups were used in AUC and duration calculations for these groups. This method is further supported as conservative since average scores remained above 50% MPE in the top doses of A2 even at the 5 hr time point as shown in FIGS. 5 and 6.

Hot Plate (HP) Test: The HP test, which reflects a supraspinally organized complex response (Chapman et al., 1985), was used to assess the CNS activation of antinociception by peripherally (sc) administered compounds. In preliminary studies, it was found that DBA mice showed HP responses at lower doses of all compounds tested, including morphine, than CD1 mice, and were therefore used for this test to reduce test compound requirements. The HP apparatus (HP, IITC, Woodland Hills, CA) was set to 55° C., a temperature that elicited a response after 7-9 sec. Three baseline HP latencies to rapidly lift, lick, or shake the hind paws were taken prior to drug injection. Mice were removed from the HP after a maximum of 30 sec. Mice were injected with various analogs (0-5.6 mg/kg s.c.) and tested from 30 to 300 min after injection. Data were converted to maximum possible effect (% MPE) as described for TF. Peak responses were later for the glycopeptides (45-60 min) compared to the reference compounds ZH853 and morphine (30-45 min). The average MPE at each of those respective times was therefore calculated and used as the peak response for determining ED₅₀'s. These values were used to calculate area under the curve (AUC). The duration of antinociception greater than 50% MPE was calculated as with TF.

Conditioned Place Preference (CPP): Male CD-1 mice were tested for CPP in a modified 2-chamber apparatus (Noldus, Leesburg, VA). The 20×20 cm chambers had distinct visual and tactile cues: 1) vertical striped walls with circular holes in a metal floor or 2) gray walls with clover-shaped holes. Lack of bias is shown with baseline times over all tests being 51.5% and 48.5% respectively. Eight arenas were counterbalanced for the cue pattern in the left and right chamber. Tests were conducted in 4 phases: habituation (days 1-2, 30′), baseline (day 4-5, 20′), conditioning (days 6-8, 40′), and testing (day 9, 20′). Two sessions per day were conducted at each phase (9:30 and 10:30 am) and animals were placed in the left chamber in session 1 and the right in session 2. For baseline and testing, both chambers were freely accessible. For conditioning, the animal received vehicle in the first session and was confined to one chamber, and in the second session, drug (vehicle, morphine, or A²) was administered in the other chamber. These protocols balanced exposure to the chambers, and for conditioning, minimized drug carry-over effects. CPP was determined as the change in time spent on the drug-associated side minus the time spent on that same side during baseline tests.

Statistics/Data Analysis: Data were analyzed with GRAPHPAD PRISM software (GraphPad Software, San Diego CA). Analysis of variance (ANOVA) and mixed model analyses, followed, when appropriate, by Newman-Kuels or Bonferroni tests, were used to assess group differences. Dose-response curves were analyzed with nonlinear regression models. Outliers were assessed by accepted statistical methods, e.g., Walfish 2006. Differences were considered statistically significant at p <0.05. Test compounds were coded during in vivo experiments and tested by blinded observers.

### Results and Discussion.

Three glycosylated EM1 analogs (A1, A2 and A7) were selected for TF testing in male and female CD-1 mice and compared to two reference compounds, ZH853 and morphine. In pilot studies Analogs A3-A6 showed TF and hot plate responses below the reference compounds and were not further tested at this time.

FIG. 5 shows the time-course of antinociception in the tail flick test, and FIG. 6 shows the area under the curve (AUC) and duration (% MPE >50%) of antinociception. Three-way analyses (drug×dose×sex) of the AUC for each pair of compounds showed the expected significant effects of dose (p<0.0001) for all drugs, reflecting significant dose dependence. Significant effects of drug were also observed (p<0.0001), reflecting the overall rank order of effectiveness as A2>A7>A1>morphine >ZH853. A main effect of sex (p<0.05) was observed in the A1 versus A2 comparison, reflecting the lower overall values for females for these two compounds. The A1 versus A7 comparison revealed a dose×drug by sex interaction (p<0.05) reflecting the sharper increase in female scores for A7, including scores greater than males at the top dose. However, two-way analyses of dose×sex for each compound showed no significant main effect of sex for the glycopeptides. A7 showed a dose×sex effect (p<0.01), confirming the steep rise in responsiveness by females described above. A significant effect sex (p<0.05) was seen in the 3 way comparison of morphine and ZH853, with subsequent 2-way drug×dose analyses a showing a significant effect (p<0.05) and a statistical tendency (p<0.1) of sex differences for morphine and ZH853 respectively. Similar statistical effects were observed for duration >50% MPE.

In summary, overall significant differences among the compounds showed a rank order of effectiveness as Analogs A2 (Lac) >A7 (Glc) >A1 (Glc) >morphine >ZH853. The effectiveness and duration of antinociception for A2 males and females, and A7 females, is a conservative estimate since several animals in each group had % MPE scores >50% at the maximum test time at 5h. To determine relative effectiveness more accurately, a separate group of mice (n=6) was tested with 10 mg/kg morphine and compared to 1.8 mg/kg A2, doses producing full (100% MPE) antinociception and a duration ≥3 hr. FIG. 6, panel B, shows that these doses produce well-matched time course data. Panel F provides AUC data versus morphine at equiantinociceptic doses. The data indicate that Analog A2 produces total antinociceptive effects equal to morphine at doses 5.55-fold to 11-fold lower on a mg/kg basis and up to 21.8-fold lower on a molar basis.

Effects of sex reflected generally lower scores for females. This occurred across doses for A1 and ZH853, while for A2, A7, and morphine, female scores were lower at low doses but closer to males at high doses. For Analog A7, female scores showed a steep increase and exceeded those of males at 5.6 mg/kg, reflected by a significant drug×dose×sex interaction in the comparison of Analogs A1 and A7.

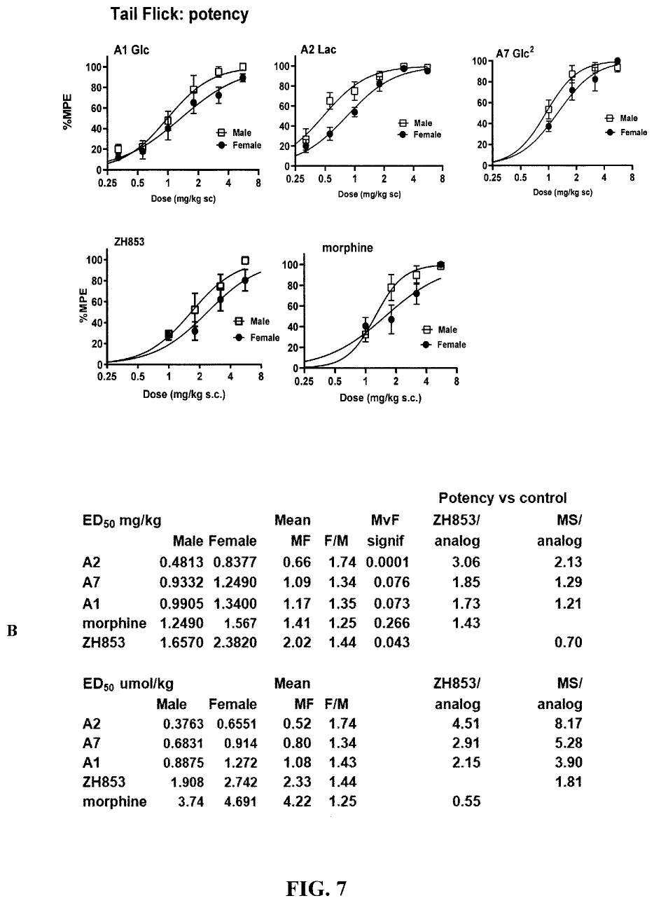

Dose-response curves and relative potency (ED₅₀) of the compounds is shown in FIG. 7. The rank order of potency is the same as that of the effectiveness/total antinociception described above (A2>A7>A1>morphine >ZH853) for mg/kg calculations, but on a molar basis (mol/kg), the rank order of morphine and ZH853 are reversed. The potency was lower for females than males for all compounds, with significant effects for Analog A2 and ZH853. None of the ratios, however, exceeded 2-fold. For both males and females, Analog A2 was significantly more potent (p<0.01-0.0001) than all other compounds except for male A2 versus A7 showing a statistical tendency (p=0.0517). Analog A2 potency relative to controls was 2-3-fold for mg/kg and 4-8 fold on a molar basis (μmol/kg).

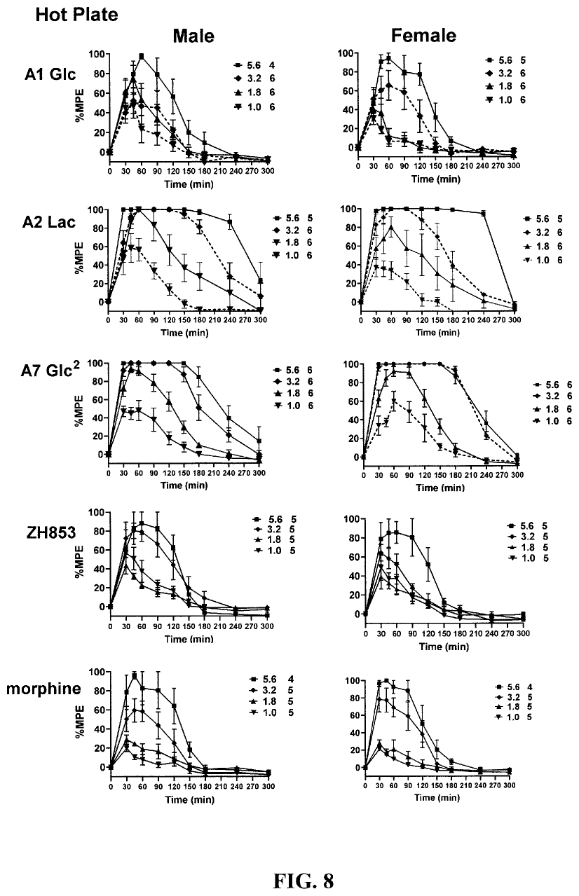

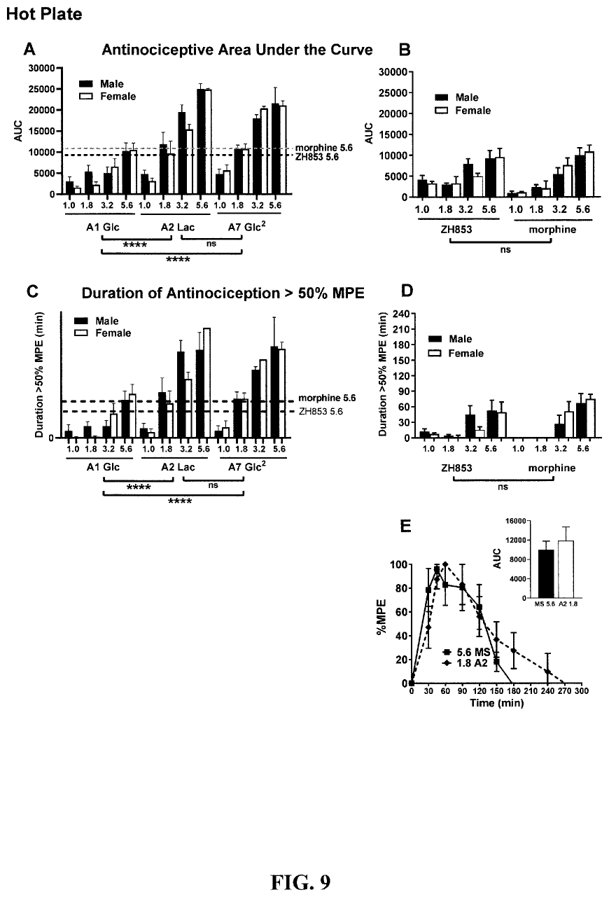

FIG. 8 shows the time course of antinociception in the HP and FIG. 9 shows the AUC (A and B) and duration (C and D). As with the tail flick test, analogs A2 and A7 produced robust antinociception in this test, consistent with the concept that these compounds activate central mechanisms regulating pain. In contrast to the TF test, however, the responses of animals given Analog A1 were not significantly greater than those of reference compounds ZH853 and morphine, and no significant sex differences were observed.

Three-way analyses (drug×dose×sex) of hot plate (HP) area under the curve (AUC) data for each pair of compounds confirmed dose-dependence with a significant effect of dose (p<0.0001 in all cases). The effect of drug was not significant between Analog A2 versus A7 or between Analog A1, ZH853, and morphine, indicating two tiers of effectiveness with Analogs A2 and A7 producing greater antinociceptive effects than the other 3 compounds. A significant effect of dose×drug for Analogs A2 or A7 compared to the other compounds indicated a greater rate of increase in response to increased dose. Sex differences (and interactions) were not significant, indicating similar effects for both sexes for all compounds in this test. One-way analyses at each dose across groups showed that Analogs A2 and A7 produced a significantly greater AUC than Analog A1, morphine and ZH853 at 3 of the four doses tested: 1.8, 3.2, and 5.6 mg/kg. As shown in FIG. 9, the AUC ranges for 1.8 mg/kg of Analogs A2 and A7 were similar to those of 5.6 mg/kg of Analog A1, ZH853, and morphine, indicating approximately 3-fold greater effectiveness of Analogs A2 and A7. FIG. 9, Panel E, shows this comparison directly as 1.8 mg/kg of Analog A2 and 5.6 mg/kg morphine produce similar peak effects, time course, and area under the curve, supporting the estimate of 3-fold greater effectiveness of Analog A2. Duration >50% MPE (C and D) showed similar statistical effects.

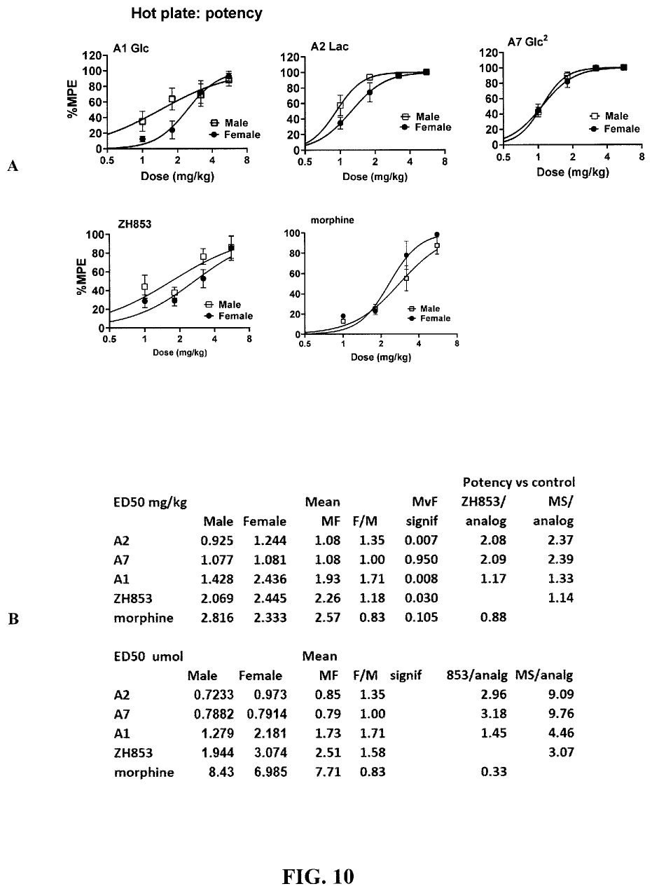

Dose-response curves and relative potency (ED₅₀) of the compounds is shown in FIG. 10. The rank order of potency is the same as that of the effectiveness/total antinociception described above for TF except that ZH853 was more potent than morphine for both mg/kg and μmol/kg values (A2>A7>A1>ZH853>morphine). The potency was lower for females than males for all compounds, with significant effects for A1, A2 and ZH853. None of the ratios, however, exceeded 2-fold. Males and females given Analogs A2 and A7, but not Analog A1, showed significantly greater potency that the ZH853 and morphine controls.

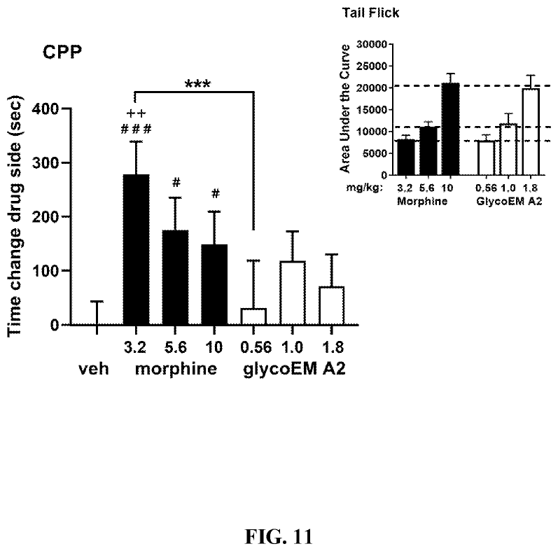

FIG. 11 shows conditioned place preference (CPP) results with 3 equiantinociceptive doses of morphine and A2, providing graphs of both time change (left) and area under curve (AUC; right inset) results. Lack of CPP is an indication that a drug may be useful for treating OUD. After two habituation and two baseline sessions, three conditioning days were conducted. Vehicle, morphine, or A2 were administered s.c. and the mouse was confined to one side of the apparatus as described in methods. The following day, a test session was conducted and CPP was determined as the change in time spent on the drug-associated side minus the time spent on that same side during baseline tests. The inset shows the 3 doses of each compound that provide equiantinociceptive effects (dashed lines) in the tail-flick test. Tests of reward often show biphasic effects, where higher doses do not produce higher effects. Here, the lowest morphine dose tested produced the highest CPP. Morphine, but not A2, produced CPP as confirmed by 1) a 2-way mixed effects analysis showing significant drug (p<0.01) and drug×dose (p<0.05) effects, and a significant difference between the 2 low doses (***, p<0.001). 2) a one-way analysis showing 3.2 mg/kg morphine greater than vehicle (++, p<0.01), and 3) t-tests that show all three morphine doses, but none of the analog doses, were significantly greater than zero (#, p<0.5) and n=12.

### REFERENCES

Al-Hasani, R., and M. R. Bruchas. 2011. ‘Molecular mechanisms of opioid receptor-dependent signaling and behavior’, Anesthesiology, 115: 1363-81.
Altarifi, A. A., B. David, K. H. Muchhala, B. E. Blough, H. Akbarali, and S. S. Negus. 2017. ‘Effects of acute and repeated treatment with the biased mu opioid receptor agonist TRV130 (oliceridine) on measures of antinociception, gastrointestinal function, and abuse liability in rodents’, J Psychopharmacol, 31: 730-9.
Araldi, D., L. F. Ferrari, and J. D. Levine. 2018. ‘Mu-opioid receptor (MOR) biased agonists induce biphasic dose-dependent hyperalgesia and analgesia, and hyperalgesic priming in the rat’, Neuroscience, 394: 60-71.
Bilsky, E. J., R. D. Egleton, S. A. Mitchell, M. M. Palian, P. Davis, J. D. Huber, H. Jones, H. I. Yamamura, J. Janders, T. P. Davis, F. Porreca, V. J. Hruby, and R. Polt. 2000. ‘Enkephalin glycopeptide analogues produce analgesia with reduced dependence liability’, Journal of Medicinal Chemistry, 43: 2586-90.
Biondi, B., E. Giannini, L. Negri, P. Melchiorri, R. Lattanzi, F. Rosso, L. Ciocca, and R. Rocchi. 2006. ‘Opioid peptides: synthesis and biological activity of new endomorphin analogues’, International Journal of Peptide Research and Therapeutics, 12: 145-51.
Bohn, L. M., R. R. Gainetdino, T. D. Sotnikova, I. O. Medvedev, R. J. Lefkowitz, L. A. Dykstra, and M. G. and Caron. 2003. ‘Enhanced rewarding properties of morphine, but not cocaine, in Barrestin knockout mice’, Journal of Neuroscience, 23:10265-73.
Bohn, L. M., R. J. Lefkowitz, R. R. Gainetdinov, K. Peppel, M. G. Caron, and F. T. Lin. 1999. ‘Enhanced morphine analgesia in mice lacking β-arrestin 2’, Science, 286:2495-8.
CDC1. ‘The three waves of opioid overdose deaths’, accessible at the cdc website cdc(dot)gov/drugoverdose/epidemic/index.
CDC2. ‘Drug overdose deaths’ accessible at the cdc website cdc(dot)gov/drugoverdose/data/statedeaths.
Chapman, C. R., K. L. Casey, R. Dubner, K. M. Foley, R. H. Gracely, and A. E. Reading. 1985. ‘Pain measurement: an overview’, Pain, 22:1-31.
Chen, G., R. G. Xie, Y. J. Gao, Z. Z. Xu, L. X. Zhao, S. Bang, T. Berta, C. K. Park, M. Lay, W. Chen, and R. R. Ji. 2016. ‘β-arrestin-2 regulates NMDA receptor function in spinal lamina II neurons and duration of persistent pain’, Nat Commun, 7:12531.
Cicero, T. J., M. S. Ellis, H. L. Surratt, and S. P. Kurtz. 2014. ‘The changing face of heroin use in the United States: a retrospective analysis of the past 50 years’, JAMA Psychiatry, 71:821-6.
Comer, S. D., M. A. Sullivan, and E. A. Walker. 2005. ‘Comparison of intravenous buprenorphine and methadone self-administration by recently detoxified heroin-dependent individuals’, Journal of Pharmacology and Experimental Therapeutics, 315:1320-30.
Czapla, M. A., D. Gozal, O. A. Alea, R. C. Beckerman, and J. E. Zadina. 2000. ‘Differential cardiorespiratory effects of endomorphin 1, endomorphin 2, DAMGO, and morphine’, American Journal of Respiratory and Critical Care Medicine, 162:994-9.
Dekan, Z., S. Sianati, A. Yousuf, K. J. Sutcliffe, A. Gillis, C. Mallet, P. Singh, A. H. Jin, A. M. Wang, S. A. Mohammadi, M. Stewart, R. Ratnayake, F. Fontaine, E. Lacey, A. M. Piggott, Y. P. Du, M. Canals, R. B. Sessions, E. Kelly, R. J. Capon, P. F. Alewood, and M. J. Christie. 2019. ‘A tetrapeptide class of biased analgesics from an Australian fungus targets the μ-opioid receptor’, Proceedings of the National Academy of Sciences of the United States of America, 116:22353-8.
Feehan, A. K., J. Morgenweck, X. Zhang, A. T. Amgott-Kwan, and J. E. Zadina. 2017. ‘Novel endomorphin analogs are more potent and longer-lasting analgesics in neuropathic, inflammatory, postoperative, and visceral pain relative to morphine’, Journal of Pain, 18:1526-41.
Ferrini, F., T. Trang, T. A. Mattioli, S. Laffray, T. Del'Guidice, L. E. Lorenzo, A. Castonguay, N. Doyon, W. Zhang, A. G. Godin, D. Mohr, S. Beggs, K. Vandal, J. M. Beaulieu, C. M. Cahill, M. W. Salter, and Y. De Koninck. 2013. ‘Morphine hyperalgesia gated through microglia-mediated disruption of neuronal Cl-homeostasis’, Nature Neuroscience, 16:183-92.
Fichna, J., M. Mazur, D. Grzywacz, W. Kamysz, R. Perlikowska, J. Piekielna, M. Sobczak, M. Salaga, G. Toth, A. Janecka, C. Chen, and J. Olczak. 2013. ‘Novel glycosylated endomorphin-2 analog produces potent centrally-mediated antinociception in mice after peripheral administration’, Bioorganic and Medicinal Chemistry Letters, 23:6673-6.
Goodman, H., A. Nix, H. Janis, S. Spröber, L. Szabo, J. Streicher, and R. Polt. “Design and synthesis of stablized, brain penetrant oxytocin agonists (unpublished work).”
Hruby, V. J., and C. A. Gehrig. 1989. ‘Recent developments in the design of receptor specific opioid peptides’, Medicinal Research Reviews, 9:343-401.
Informatics, IMS Institute for Healthcare. 2014. “Medicine use and shifting costs of healthcare. A review of the use of medicines in the United States in 2013.”
Isidro-Llobet, A., M. Alvarez, and F. Albericio. 2009. ‘Amino acid-protecting groups’, Chemical Reviews, 109:2455-504.
Jones, J. D., G. Madera, and S. D. Comer. 2014. ‘The reinforcing and subjective effects of intravenous and intranasal buprenorphine in heroin users’, Pharmacology, Biochemistry and Behavior, 122:299-306.
Lefever, M., Y. Li, B. Anglin, D. Muthu, D. Giuvelis, J. J. Lowery, B. I. Knapp, J. M. Bidlack, E. J. Bilsky, and R. Polt. 2015. ‘Structural requirements for CNS active opioid glycopeptides’, Journal of Medicinal Chemistry, 58:5728-41.
Li, Y., M. R. Lefever, D. Muthu, J. M. Bidlack, E. J. Bilsky, and R. Polt. 2012. ‘Opioid glycopeptide analgesics derived from endogenous enkephalins and endorphins’, Future Medicinal Chemistry, 4:205-26.
Li Y, St Louis L, Knapp B I, Muthu D, Anglin B, et al. Can amphipathic helices influence the CNS antinociceptive activity of glycopeptides related to β-endorphin?. J Med Chem. 2014 Mar. 27; 57 (6): 2237-46. PubMed PMID: 24576160.
Lowery, J. J., L. Yeomans, C. M. Keyari, P. Davis, F. Porreca, B. I. Knapp, J. M. Bidlack, E. J. Bilsky, and R. Polt. 2007. ‘Glycosylation improves the central effects of DAMGO’, Chemical Biology & Drug Design, 69:41-7.
Mabrouk, O. S., T. Falk, S. J. Sherman, R. T. Kennedy, and R. Polt. 2012. ‘CNS penetration of the opioid glycopeptide MMP-2200: a microdialysis study’, Neuroscience Letters, 531:99-103.
Martin, T. J., S. A. Kim, N. L. Buechler, F. Porreca, and J. C. Eisenach. 2007. ‘Opioid self-administration in the nerve-injured rat. Relevance of antiallodynic effects to drug consumption and effects of intrathecal analgesics’, Anesthesiology, 106:312-22.
Mitchell, S. A.; Pratt, M. R.; Hruby, V. J.; Polt, R. Solid-Phase Synthesis of O-Linked Glycopeptide Analogues of Enkephalin. J. Org. Chem. 2001, 66, 2327-2342.
Mosberg, H. I., L. Yeomans, J. P. Anand, V. Porter, K. Sobczyk-Kojiro, J. R. Traynor, and E. M. Jutkiewicz. 2014. ‘Development of a bioavailable μ opioid receptor (MOPr) agonist, δ opioid receptor (DOPr) antagonist peptide that evokes antinociception without development of acute tolerance’, Journal of Medicinal Chemistry, 57:3148-53.
NHTSA ‘Motor vehicle traffic crashes’ accessible at the NHTSA website at www(dot)fars.nhtsa.dot(dot)gov/Main/index.
Nilges, M. R., M. Laurent, C. Cable, L. Arens, J. Vafiades, and J. E. Zadina. 2019. ‘Discriminative stimulus and low abuse liability effects of novel endomorphin analogs suggest a potential treatment indication for opioid use disorder’, Journal of Pharmacology and Experimental Therapeutics, 370:369-79.
O'Connor, E. C., K. Chapman, P. Butler, and A. N. Mead. 2011. ‘The predictive validity of the rat self-administration model for abuse liability’, Neuroscience and Biobehavioral Reviews, 35:912-38.
Oprea, T. I., C. G. Bologa, S. Brunak, A. Campbell, G. N. Gan, A. Gaulton, S. M. Gomez, R. Guha, A. Hersey, J. Holmes, A. Jadhav, L. J. Jensen, G. L. Johnson, A. Karlson, A. R. Leach, A. Ma'ayan, A. Malovannaya, S. Mani, S. L. Mathias, M. T. McManus, T. F.
Polt R, Szabò L Z, Treiberg J, Li Y, Hruby V J. General methods for α- or β-O-Ser/Thr glycosides and glycopeptides. Solid-phase synthesis of O-glycosyl cyclic enkephalin analogues. J Am Chem Soc 1992, 114, 10249-10258.
Meehan, C. von Mering, D. Muthas, D. T. Nguyen, J. P. Overington, G. Papadatos, J. Qin, C. Reich, B. L. Roth, S. C. Schurer, A. Simeonov, L. A. Sklar, N. Southall, S. Tomita, I. Tudose, O. Ursu, D. Vidovic, A. Waller, D. Westergaard, J. J. Yang, and G. Zahoranszky-Kohalmi. 2018. ‘Unexplored therapeutic opportunities in the human genome’, Nat Rev Drug Discov, 17:317-32.
Raehal, K. M., J. K. Walker, and L. M. Bohn. 2005. ‘Morphine side effects in β-arrestin 2 knockout mice’, Journal of Pharmacology and Experimental Therapeutics, 314:1195-201.
Schmid, C. L., N. M. Kennedy, N. C. Ross, K. M. Lovell, Z. Yue, J. Morgenweck, M. D. Cameron, T. D. Bannister, and L. M. Bohn. 2017. ‘Bias factor and therapeutic window correlate to predict safer opioid analgesics’, Cell, 171:1165-75.
Steinpreis, R. E., A. L. Rutell, and F. A. Parrett. 1996. ‘Methadone produces conditioned place preference in the rat’, Pharmacology, Biochemistry and Behavior, 54:339-41.
Stevenson, G. W., D. Giuvelis, J. Cormier, K. Cone, P. Atherton, R. Krivitsky, E. Warner, B. St Laurent, J. Dutra, J. M. Bidlack, L. Szabo, R. Polt, and E. J. Bilsky. 2020. ‘Behavioral pharmacology of the mixed-action delta-selective opioid receptor agonist BBI-11008: studies on acute, inflammatory and neuropathic pain, respiration, and drug self-administration’, Psychopharmacology.
Thieret, N.; Alsina, J.; Giralt, E.; Guibe, F.; Albericio, F. Use of Alloc-amino acids in solid-phase peptide synthesis. Tandem deprotection-coupling reactions using neutral conditions. Tetrahedron Lett. 1997, 38 (41), 7275-7278.
Thompson, G. L., J. R. Lane, T. Coudrat, P. M. Sexton, A. Christopoulos, and M. Canals. 2016. ‘Systematic analysis of factors influencing observations of biased agonism at the mu-opioid receptor’, Biochemical Pharmacology, 113:70-87.
Tzschentke, T. M. 2004. ‘Reassessment of buprenorphine in conditioned place preference: temporal and pharmacological considerations’, Psychopharmacology, 172:58-67.
Varamini, P., F. M. Mansfeld, J. T. Blanchfield, B. D. Wyse, M. T. Smith, and I. Toth. 2012. ‘Synthesis and biological evaluation of an orally active glycosylated endomorphin-1’, Journal of Medicinal Chemistry, 55:5859-67.
Wade, C L, L F Vendruscolo, J E Schlosburg, D O Hernandez, and G. F. and Koob. 2015. ‘Compulsive-like responding for opioid analgesics in rats with extended access’, Neuropsychopharmacology, 40:421-8.
Walfish, S. 2006. ‘A review of statistical outlier methods’, Pharm Tech, 30:82-6.
Wang, Z., W. Ma, J. G. Chabot, and R. Quirion. 2010. ‘Calcitonin gene-related peptide as a regulator of neuronal CaMKII-CREB, microglial p38-NFκB and astroglial ERK-Stat1/3 cascades mediating the development of tolerance to morphine-induced analgesia’, Pain, 151:194-205.
Watkins, L. R., M. R. Hutchinson, I. N. Johnston, and S. F. Maier. 2005. ‘Glia: novel counter-regulators of opioid analgesia’, Trends in Neurosciences, 28:661-9.
Wilson, A. M., R. D. Soignier, J. E. Zadina, A. J. Kastin, W. L. Nores, R. D. Olson, and G. A. Olson. 2000. ‘Dissociation of analgesic and rewarding effects of endomorphin-1 in rats’, Peptides, 21:1871-4.
Zadina, J. E., L. Hackler, L. J. Ge, and A. J. Kastin. 1997. ‘A potent and selective endogenous agonist for the μ-opiate receptor’, Nature, 386:499-502.
Zadina, J. E., M. R. Nilges, J. Morgenweck, X. Zhang, L. Hackler, and M. B. Fasold. 2016. ‘Endomorphin analog analgesics with reduced abuse liability, respiratory depression, motor impairment, tolerance, and glial activation relative to morphine’, Neuropharmacology, 105:215-27.
Zadina, J. E., and L. Hackler. “Mu opioid receptor agonist analogs of the endomorphins OPIOID Patent US 2017/0362276 A1 Dec. 21, 2017.”

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing materials or methods (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The terms “consisting of” and “consists of” are to be construed as closed terms, which limit any compositions or methods to the specified components or steps, respectively, that are listed in a given claim or portion of the specification. In addition, and because of its open nature, the term “comprising” broadly encompasses compositions and methods that “consist essentially of” or “consist of” specified components or steps, in addition to compositions and methods that include other components or steps beyond those listed in the given claim or portion of the specification. Recitation of ranges of values herein are merely intended to serve as a shorthand method of referring individually to each separate value falling within the range, unless otherwise indicated herein, and each separate value is incorporated into the specification as if it were individually recited herein. All numerical values obtained by measurement (e.g., weight, concentration, physical dimensions, removal rates, flow rates, and the like) are not to be construed as absolutely precise numbers, and should be considered to encompass values within the known limits of the measurement techniques commonly used in the art, regardless of whether or not the term “about” is explicitly stated. All methods described herein can be performed in any suitable order unless otherwise indicated herein or otherwise clearly contradicted by context. The use of any and all examples, or exemplary language (e.g., “such as”) provided herein, is intended merely to better illuminate certain aspects of the materials or methods described herein and does not pose a limitation on the scope of the claims unless otherwise stated. No language in the specification should be construed as indicating any non-claimed element as essential to the practice of the claims.

Preferred embodiments are described herein, including the best mode known to the inventors for carrying out the claimed invention. Variations of those preferred embodiments may become apparent to those of ordinary skill in the art upon reading the foregoing description. The inventors expect skilled artisans to employ such variations as appropriate, and the inventors intend for the claimed invention to be practiced otherwise than as specifically described herein. Accordingly, the claimed invention includes all modifications and equivalents of the subject matter recited in the claims appended hereto as permitted by applicable law. Moreover, any combination of the above-described elements in all possible variations thereof is encompassed by the claimed invention unless otherwise indicated herein or otherwise clearly contradicted by context.

We claim:

1. A glycosylated, cyclic peptide of Formula (I): A¹-cyclo[A²-A³-A⁴-A⁵]-A⁶-O-Carb, Formula (II): A¹-cyclo[A⁵-A³-A⁴-A²]-A⁶-O-Carb, and pharmaceutically acceptable salts thereof,
wherein:
A¹ is an amino acid residue selected from the group consisting of Tyr, N-methyl-Tyr, 2,6-dimethyl-Tyr, and N-methyl-2,6-dimethyl-Tyr;
A² is an amino acid residue selected from the group consisting of Lys, hLys, bhLys, Orn, Dab, and Dpr;
A³ is an amino acid residue selected from the group consisting of Trp, N-methyl-Trp, Phe, N-methyl-Phe, p-X-Phe, and N-methyl-p-X-Phe;
A⁴ is an amino acid residue selected from the group consisting of Phe, N-methyl-Phe, p-X-Phe, and N-methyl-p-X-Phe;
X is F, Cl, Br, or NO₂ at the 4-position of the phenyl group of the Phe sidechain;
A⁵ is an amino acid residue selected from the group consisting of Asp, Glu, homoGlu, bishomoGlu, isoAsp, isoGlu, isohomoGlu, and isobishomoGlu;
A⁶ is an amino acid residue or an oligopeptide comprising 2 to 5 amino acid residues, and A⁶ comprises at least one hydroxy-substituted amino acid residue;
Carb is a carbohydrate group bonded to a sidechain oxygen of the HO-AA by a β-D-glycosidic bond;
cyclo[A²-A³-A⁴-A⁵] and cyclo[A²-A³-A⁴-A⁵] each represent a cyclic peptide ring in which A² is bonded to A⁵; wherein when A⁵ is isoAsp, isoGlu, isohomoGlu, or isobishomoGlu, the sidechain amino group of A² is bonded the α-carboxyl group of A⁵ by an amide bond; whereas when A⁵ is Asp, Glu, homoGlu or bishomoGlu, the sidechain amino group of A² is bonded to the sidechain carboxyl group of A⁵ by an amide bond; and
wherein the C-terminal carboxyl group of A⁶ optionally is amidated.

2. The peptide of claim 1, wherein A¹ is Tyr.

3. The peptide of claim 1, wherein A¹ is N-methyl-Tyr.

4. The peptide of any one of claims 1 to 3, wherein A³ is Trp.

5. The peptide of any one of claims 1 to 3, wherein A³ is N-methyl-Trp.

6. The peptide of any one of claims 1 to 3, wherein A³ is Phe.

7. The peptide of any one of claims 1 to 6, wherein A⁴ is Phe.

8. The peptide of any one of claims 1 to 6, wherein A⁴ is N-methyl-Phe.

9. The peptide of any one of claims 1 to 6, wherein A⁴ is p-X-Phe, wherein X is F, Cl, Br, or NO₂ at the 4-position of the phenyl group of the Phe sidechain.

10. The peptide of any one of claims 1 to 6, wherein A⁴ is N-methyl-p-X-Phe, wherein X is F, Cl, Br, or NO₂ at the 4-position of the phenyl group of the Phe sidechain.

11. The peptide of claim 1, wherein A¹ is Tyr, A³ is Trp, and A⁴ is Phe.

12. The peptide of any one of claims 1 to 11, wherein A⁵ is L-Asp.

13. The peptide of any one of claims 1 to 11, wherein A⁵ is L-Glu.

14. The peptide of any one of claims 1 to 11, wherein A⁵ is D-Asp.

15. The peptide of any one of claims 1 to 11, wherein A⁵ is D-Glu.

16. The peptide of any one of claims 1 to 11, wherein A⁵ is L-isoAsp.

17. The peptide of any one of claims 1 to 11, wherein A⁵ is L-isoGlu.

18. The peptide of any one of claims 1 to 11, wherein A⁵ is D-isoAsp.

19. The peptide of any one of claims 1 to 11, wherein A⁵ is D-isoGlu.

20. The peptide of any one of claims 1 to 19, wherein A⁶ is L-Ser.

21. The peptide of any one of claims 1 to 19, wherein A⁶ is L-Ser-NH₂.

22. The peptide of any one of claims 1 to 19, wherein A⁶ is L-Thr.

23. The peptide of claim 1, wherein A⁶ is L-Thr-NH₂.

24. The peptide of any one of claims 1 to 23, wherein Carb is β-D-glucose.

25. The peptide of any one of claims 1 to 23, wherein Carb is β-D-lactose.

26. The peptide of any one of claims 1 to 25, where in the peptide is a cyclic peptide of Formula (I).

27. The peptide of any one of claims 1 to 25, where in the peptide is a cyclic peptide of Formula (II).

28. The peptide of claim 1 or claim 26 which is:
(SEQ ID NO: 1)
Tyr-c[D-Lys-Trp-Phe-Glu]-Ser(β-Lact)-NH₂.

29. The peptide of claim 1 or claim 26 which is:
(SEQ ID NO: 5)
Tyr-c[D-Lys-Trp-Phe-Glu]-Ser(β-Glc)-NH₂.

30. The peptide of claim 1 or claim 26 which is:
(SEQ ID NO: 10)
Tyr-c[D-Lys-Trp-Phe-Glu]-Ser(β-Glc)-Ser(β-Glc)-NH₂.

31. A pharmaceutical composition comprising the peptide of any one of claims 1 to 30 or a pharmaceutically acceptable salt thereof in a pharmaceutically acceptable carrier.

32. A method of treating pain comprising administering to a subject in need thereof the pharmaceutical composition of claim 31.

33. The method of claim 32, wherein the pain is chronic pain.

34. The method of claim 32 or claim 33, wherein the pain is neuropathic pain.

35. The method of any one of claim 32 to claim 34, wherein the pain is inflammatory pain.

36. A method for treating a drug dependence comprising administering to a subject in need thereof a therapeutically effective amount of the pharmaceutical composition of claim 31.

37. A method for treating opioid use disorder comprising administering to a subject in need thereof a therapeutically effective amount of the pharmaceutical composition of claim 31.

38. The method of claim 37, wherein the composition is administered in place of, and as a substitute for, a mu opioid receptor agonist to which the subject is addicted.

39. The peptide of any one of claims 1 to 30 for treating pain.

40. The peptide of claim 39, wherein the pain is chronic pain.

41. The peptide of claim 39 or claim 40, wherein the pain is neuropathic pain.

42. The peptide of any one of claim 39 to claim 41, wherein the pain is inflammatory pain.

43. The peptide of any one of claims 1 to 30 for treating drug dependence.

44. The peptide of any one of claims 1 to 30 for treating opioid use disorder.
